(19) United States
(12) Patent Application Publication
(10) Pub. No.: US 2017/0360283 A1
(43) Pub. Date: Dec. 21, 2017

(54) CAPSULE ENDOSCOPE, CAPSULE ENDOSCOPIC INSPECTION METHOD, AND CAPSULE ENDOSCOPIC INSPECTION DEVICE

(71) Applicants: PIOLAX, INC., Yokohama-shi (JP); KYUSHU INSTITUTE OF TECHNOLOGY, Kitakyushu-shi (JP)

(72) Inventors: Toshihiro KIMURA, Yokohama-shi, Kanagawa (JP); Takahiro ITO, Kitakyushu-shi, Fukuoka (JP)

(21) Appl. No.: 15/535,366

(22) PCT Filed: Dec. 16, 2015
(86) PCT No.: PCT/JP2015/085249

§ 371 Date: Jun. 12, 2017

(30) Foreign Application Priority Data
Dec. 18, 2014 (JP) 2014-256653
Sep. 16, 2015 (JP) 2015-182501

(51) Int. Cl. A61B 1/04; A61B 1/00; H01F 38/14; H02J 50/10; A61B 1/06; H04N 5/225

(52) U.S. Cl. CPC A61B 1/041; A61B 1/06; H04N 5/2256; H01F 38/14; H02J 50/10; A61B 1/00147; A61B 1/00029; A61B 1/00016; H04N 2005/2255

(57) ABSTRACT

A capsule endoscope according to one embodiment includes: a camera; a transceiver; a tubular receiving coil for receiving power supplied from an external power transmitting antenna via magnetic flux; a tubular capsule accommodating these components; and an X-ray marker to be used in location and orientation detection. In the capsule endoscope, a magnetic body is arranged along the inner periphery of the receiving coil, and a self-propelling drive device including an electromagnet and a permanent magnet is arranged in series with the receiving coil along the tubular axial direction of the capsule so that the permanent magnet does not enter the inside of the receiving coil.

### TECHNICAL FIELD

The present invention relates to a generally cylindrical capsule endoscope that is put into tubular organs such as the digestive organs and used for diagnosing the condition of the inside of the tubular organs, as well as to a capsule endoscope examining method and a capsule endoscope examination instrument.

### BACKGROUND ART

Capsule endoscopes, which are devices for observing the digestive organs from inside by ingesting the device that is shaped like a small capsule and incorporates a camera, are superior in enabling observation of the entire route from the mouth to the anus, particularly a deep part of the small intestine which is difficult to observe with fiber endoscopes, while causing subjects to feel almost no physical loads. Images taken from inside the digestive organs are transmitted sequentially to the outside by a wireless communication and displayed on a monitor, whereby a diagnosis is enabled.

Since capsule endoscopes are moved by vermicular movements of the digestive organs, it takes several hours for the capsule endoscope to be ejected (from its ingestion). Capsule endoscopes incorporate a battery to drive electronic components such as a camera during that period.

However, since capsule endoscopes are restricted in size so as to be easy to digest, battery-type capsule endoscopes have no room for devices other than a camera. Furthermore, batteries can supply only limited power and there are limitations on the functions that can be implemented through driving by such limited power.

For the above reasons, wireless power supply methods for supplying power from outside by electromagnetic induction are being studied. For example, the following Patent document 1 discloses a capsule endoscope that is equipped with a power-reception coil, storage batteries that are disposed in the power-reception coil, have such a size as to occupy the inside of the power-reception coil, and are each covered with a metal case and electrodes, a function performing unit to perform prescribed functions, and a core member that is disposed so as to penetrate through the power-reception coil in its axial direction, extends a prescribed length at both ends in the longitudinal direction perpendicularly to the axial direction of the power-reception coil, and changes the direction of at least part of a magnetic flux directed to the function performing unit so that that (part of) the magnetic flux goes into the core member itself.

Patent document 2 discloses, as an example configuration to supply power to a capsule endoscope, a cylindrical power-transmission coil that is disposed inside clothes to be worn by a subject and is to be wound around his or her torso.

### PRIOR ART DOCUMENTS

### Patent Documents

### PATENT DOCUMENT 1: JP-4624768-B

### PATENT DOCUMENT 2: JP-5356697-B

### SUMMARY OF THE INVENTION

### Problems to be Solved by the Invention

However, capsule endoscopes that are configured so as to be supplied with power from outside by electromagnetic induction have two major problems that relate to securing of power.

The first problem is that where a conductor having such a size as to occupy the inside of a power-reception coil is disposed inside the power-reception coil, an eddy current is generated in the conductor by electromagnetic induction, resulting in power loss. The second problem is that when the position or posture of a capsule endoscope with respect to a transmission antenna is changed, the density of an interlinkage magnetic flux may decrease to lower the power reception efficiency.

In Patent document 1, the influence of an eddy current is reduced by changing the direction of a magnetic flux by the core member. However, since the conductors, having such a size as to occupy the inside of a power-reception coil, of the storage batteries are disposed inside the power-reception coil, increase of received power is not attained.

In Patent document 2, the variation of the strength of a magnetic field depending on the distance from the power-transmission coil is suppressed by an auxiliary coil. However, it has no disclosure as to a method for varying the direction of the magnetic field according to the posture of the capsule endoscope.

An object of the present invention is therefore to provide a capsule endoscope that is high in power reception efficiency, an examining method using that capsule endoscope, and a capsule endoscope examination instrument capable of supplying power to the capsule endoscope efficiently even when its position or posture is changed.

### Means for Solving the Problems

To attain the above object, the invention provides

a generally cylindrical capsule endoscope to be used for diagnosing the condition of the inside of a tubular organ such as a digestive tract by going into the tubular organ, the capsule endoscope including:

a camera which shoots the inside of the tubular organ;

a transceiver which performs a wireless communication with the outside;

a cylindrical power-reception coil which receives power that is supplied from an external power-transmission antenna via a magnetic flux;

a self-propulsion drive device which causes the capsule endoscope to move along the inside of the tubular organ; and

a generally cylindrical capsule which houses the above components,

wherein a magnetic body is disposed adjoining an inner circumferential surface of the power-reception coil,

wherein the self-propulsion drive device has a coil and a magnet, and

wherein the self-propulsion drive device is disposed in series to the power-reception coil in an axial direction of the capsule so as not to be located inside the power-reception coil.

According to the above mode, efficient power reception is enabled because the magnetic body which is disposed adjoining the inner circumferential surface of the power-reception coil increases the density of a magnetic flux that interlinks with the power-reception coil. Received AC power is converted into DC power which is supplied to the camera, the transceiver, etc. This makes it possible to shoot various portions of the tubular organ and send images thus taken to the outside wirelessly. Since the self-propulsion drive device is disposed in series to the power-reception coil in the axial direction of the capsule so that the permanent magnet is not located inside the power-reception coil, the effect that the magnetic body which is disposed adjoining the inner circumferential surface of the power-reception coil enhances the magnetic flux density is not impaired. Furthermore, since other components can be disposed inside the power-reception coil, the space in the capsule can be utilized effectively. Still further, since the self-propulsion drive device allows the capsule endoscope to move along the inside of a tubular organ and shoot the inside of the tubular organ, examination in a wide range can be performed in a short time.

There may be provided

the capsule endoscope,

wherein a central portion and both end portions of the capsule are cylindrical and hemispherical, respectively, an outer circumferential surface of the cylindrical portion is formed with a ring-shaped recess, the magnetic body is disposed at the bottom of the recess, the power-reception coil is disposed adjoining an outer circumferential surface of the magnetic body, and the magnetic body and the power-reception coil are housed within a wall thickness of the capsule.

There may be provided

the capsule endoscope,

wherein the magnetic body is one formed by curling a resin sheet of 0.1 to 0.5 mm in thickness containing a ferromagnetic material.

According to the above modes, by forming a thin cylindrical magnetic body, efficient power reception is enabled in a state that a wide space is left inside the power-reception coil. And it becomes possible to utilize the space inside the power-reception coil effectively by disposing various members there and to thereby miniaturize the capsule.

There may be provided

the capsule endoscope,

wherein the capsule is 0.5 to 1.0 mm in thickness, the magnetic body is one formed by curling a resin sheet of 100 to 130 in relative permeability and 0.2 to 0.3 mm in thickness containing a ferromagnetic material, and the power-reception coil is a cylindrical coil of 4 to 6.5 mm in coil length formed by winding a coated wire of 0.10 to 0.15 mm in outer diameter around an outer circumferential surface of the magnetic body in two layers.

According to the above mode, the magnetic body and the power-reception coil be housed within the wall thickness of the capsule, which enables efficient power reception.

There may be provided

the capsule endoscope,

wherein a curled electronic circuit board which controls installed devices is disposed in the capsule.

According to the above mode, by housing even an electronic circuit board having a wide area inside the power-reception coil in a curled state, it becomes possible to utilize the space effectively and thereby miniaturize the capsule.

There may be provided

the capsule endoscope,

wherein a central portion and both end portions of the capsule are cylindrical and hemispherical, respectively, an outer circumferential surface of a hemispherical portion that is opposite to an end portion where the camera is disposed is formed with a ring-shaped recess, the magnetic body is disposed at the bottom of the recess, the power-reception coil is disposed adjoining an outer circumferential surface of the magnetic body, and the magnetic body and the power-reception coil are housed in the hemispherical portion of the capsule.

There may be provided

the capsule endoscope,

wherein the magnetic body is one formed by curling a resin sheet of 100 to 130 in relative permeability and 0.1 to 0.5 mm in thickness containing a ferromagnetic material, and the power-reception coil is a cylindrical coil of 3 to 4 mm in coil length formed by winding a coated wire of 0.10 to 0.15 mm in outer diameter around an outer circumferential surface of the magnetic body in three or more layers.

According to the above modes, the hemispherical end portion whose empty space is difficult to utilize can be employed as a place to dispose the power-reception coil and the magnetic body, whereby high permeability components and conductive components can be disposed in the space inside the cylindrical portion of the capsule barrel in a desired manner.

There may be provided

the capsule endoscope,

wherein a liquid chemical supply device is disposed inside the power-reception coil, and includes:
a non-metal liquid chemical tank;
an electromotive valve or pump which is connected to the liquid chemical tank and driven by power received by the power-reception coil; and
a liquid chemical emission opening which is formed at an end portion of the capsule.

According to the above mode, since the liquid chemical supply device is disposed inside the power-reception coil, the space can be utilized effectively and the capsule can be miniaturized. Furthermore, since the electromotive valve or pump can be driven by an external control signal, a liquid chemical can be administered at a desired time and place.

There may be provided

the capsule endoscope,

wherein a microhand device is disposed inside the power-reception coil, and device includes:
a resin shape-memorized spring which is memorized with an elongated shape at a high temperature and housed in a compressed state at low temperatures;
a ceramic heater which heats the resin shape-memorized spring when driven by power received by the power-reception coil; and
non-metal (resin or ceramic) scissors which are attached to the tip of the shape-memorized spring,

wherein the scissors project from an opening of an end portion of the capsule and open as a result of elongation of the shape-memorized spring that is caused by energization of the ceramic heater, and

wherein when the ceramic heater is deenergized, the shape-memorized spring cools and, during that course, the scissors are pulled back and closed as they come to be confined in the opening of the end portion of the capsule.

According to the above mode, since the microhand device is disposed in the space inside the cylindrical power-reception coil, the space can be utilized effectively and the capsule can be miniaturized. Furthermore, since the microhand device employed in the invention has such a simple structure as to be opened and closed when the heater is energized and deenergized, respectively, using a sufficient amount of power received efficiently, the microhand device has features that it is small and less likely to fail. Furthermore, capable of being driven by an external control signal, tract tissue can be sampled at a desired time and place.

The embodiment also provides a capsule endoscope examining method which supplies power intermittently to the power-reception coil of the capsule endoscope having any of the above-described configurations.

According to the above mode, power is supplied to the power-reception coil intermittently, which enables an operation as exemplified below. During periods when power is supplied to the power-reception coil, the capsule endoscope is caused to move on its own using the self-propulsion drive device. During periods when power is not supplied to the power-reception coil, the capsule endoscope is allowed to cool by deenergizing the self-propulsion drive device. The capsule can be miniaturized because no sensor or temperature control circuit for preventing temperature increase of the capsule endoscope is necessary.

The capsule endoscope examining method may include

detecting a start and an end of a period when the power-reception coil is not supplied with power by a power measurement unit which measures the magnitude of power being received by the power-reception coil or a time measurement unit which operates in synchronism with supply of power to the power-reception coil.

According to the above mode, by detecting a start and an end of a period when the power-reception coil is not supplied, with power, the capsule endoscope can be controlled in synchronism with supply of power that is performed intermittently.

The capsule endoscope examining method may include

performing a wireless communication with the outside by the transceiver in the period when the power-reception coil is not supplied with power.

According to the above mode, a communication error due to electromagnetic field noise can be avoided by performing a wireless communication in periods when the power-reception coil is not supplied with power.

The invention also provides

a capsule endoscope examination instrument which uses

a capsule endoscope including:
a camera which shoots the inside of a tubular organ;
a transceiver which performs a wireless communication with the outside;
a cylindrical power-reception coil which receives power that is supplied from an external power-transmission antenna via a magnetic flux;
a transmission unit which measures the magnitude of received power and communicates the magnitude of the received power wirelessly;
a self-propulsion drive device which causes the capsule endoscope to move along the inside of the tubular organ; and
a generally cylindrical capsule which houses the above components,
wherein a magnetic body is disposed adjoining an inner circumferential surface of the power-reception coil,
wherein the self-propulsion drive device has an electromagnet and a permanent magnet, and
wherein the self-propulsion drive device is disposed in series to the power-reception coil in an axial direction of the capsule so that the permanent magnet is not located inside the power-reception coil,

the capsule endoscope examination instrument including:

an examination stage on which a subject is placed;

a power-transmission antenna or antennas which are disposed under and/or over a subject placement part of the examination stage so as to be movable relative to the examination stage, to supply power wirelessly to the power-reception coil of the capsule endoscope;

a receiving unit which receives a signal from the transmission unit of the capsule endoscope; and

a power-transmission antenna position controller which performs a scan by moving the power-transmission antenna or antennas relative to the examination stage, and sets the power-transmission antenna or antennas at a position or positions where the power-transmission antenna or antennas allow the received power to be larger than or equal to a prescribed value.

According to the above mode, since a scan be performed by moving the power-transmission antenna or antennas relative to the examination stage and the power-transmission antenna or antennas are set at a position or positions where the power-transmission antenna or antennas allow the received power to be larger than or equal to the prescribed value, the power-transmission antenna or antennas can be set so as to secure necessary received power wherever the capsule endoscope is located.

The invention also provides

a capsule endoscope examination instrument which uses

a capsule endoscope including:
a camera which shoots the inside of a tubular organ;
a transceiver which performs a wireless communication with the outside;
a cylindrical power-reception coil which receives power that is supplied from an external power-transmission antenna via a magnetic flux;
a self-propulsion drive device which causes the capsule endoscope to move along the inside of the tubular organ;
a generally cylindrical capsule which houses the above components; and
a detector which detects a position and a posture of the capsule,
wherein a magnetic body is disposed adjoining an inner circumferential surface of the power-reception coil,
wherein the self-propulsion drive device has an electromagnet and a permanent magnet, and
wherein the self-propulsion drive device is disposed in series to the power-reception coil in an axial direction of the capsule so that the permanent magnet is not located inside the power-reception coil,

the capsule endoscope examination instrument including:

an examination stage on which a subject is placed;

a power-transmission antenna or antennas which are disposed under and/or over a subject placement part of the examination stage so as to be movable independently relative to the examination stage, to supply power wirelessly to the power-reception coil of the capsule endoscope;

a receiving unit which receives a signal from a transmission unit of the capsule endoscope;

a position determinator which determines a position or positions of the power-transmission antenna or antennas where the power-transmission antenna or antennas allow the received power to be larger than or equal to a prescribed value on the basis of the position and the posture of the capsule endoscope detected by the detector; and

a power-transmission antenna position controller which moves the power-transmission antenna or antennas on the basis of a result obtained by the position determinator.

According to the above mode, by detecting a position and a posture of the capsule endoscope by the detector, the power-transmission antenna or antennas can be set so that the reception power becomes larger than or equal to the prescribed value wherever the capsule endoscope is located. Thus, even if the posture of the capsule endoscope is changed quickly, a stable endoscope examination can be performed by securing necessary received power by moving the power-transmission antenna or antennas efficiently.

The invention also provides

the capsule endoscope examination instrument,

wherein the power-transmission antenna or antennas are ones formed by winding a conductor into a planar spiral or a coil and shaped like a circular ring having a central hole, and, when the power-transmission antenna or antennas are supplied with AC power, a divergent magnetic field that diverges outward from the central hole is formed to supply power to the capsule endoscope.

According to the above mode, since a divergent magnetic field is formed that diverges outward from the central hole of each power-transmission antenna, the power-transmission antenna or antennas can be set so that the axis of the power-reception coil is parallel with magnetic field lines, whereby efficient supply of power is enabled.

The invention also provides

the capsule endoscope examination instrument,

wherein the capsule endoscope includes a detector which detects a position and a posture of the capsule,

wherein the power-transmission antenna is disposed under or over the subject placement part of the examination stage so that it is movable relative to the examination stage and the axis of the circular ring is perpendicular to the examination stage, and

wherein,
if the axis of the power-reception coil is parallel with the axis of the circular ring of the power-transmission antenna,
   the power-transmission antenna is moved so that the capsule endoscope comes to be located inside an inner edge of the power-transmission antenna,
if the axis of the power-reception coil is parallel with a plane that is perpendicular to the axis of the circular ring of the power-transmission antenna,
   the power-transmission antenna is moved so that the capsule endoscope comes to be located near an outer edge of the power-transmission antenna and the axis of the power-reception coil is directed in a radial direction of the power-transmission antenna, and
if the axis of the power-reception coil is inclined with respect to a plane that is perpendicular to the axis of the circular ring of the power-transmission antenna,
   the power-transmission antenna is moved so that the capsule endoscope comes to be located in a region of the circular ring between the inner edge and the outer edge of the power-transmission antenna and the axis of the power-reception coil is directed in the radial direction of the power-transmission antenna.

According to the above mode, the power supply efficiency can be increased because a position and a posture of the capsule is detected by the detector and the power-transmission antenna can be moved so as to generate magnetic field lines that are parallel with the axis of the power-reception coil.

The invention also provides

the capsule endoscope examination instrument,

wherein the capsule endoscope includes a detector which detects a position and a posture of the capsule,

wherein the power-transmission antennas include a first power-transmission antenna and a second power-transmission antenna that are disposed under and over the subject placement part of the examination stage, respectively, so that they are movable relative to the examination stage and the axis of the circular ring is perpendicular to the examination stage, and

wherein,
if the axis of the power-reception coil is parallel with the axis of the circular ring of each of the first and second power-transmission antennas,
   the first and second power-transmission antennas are arranged coaxially, moved so that the capsule endoscope comes to be located inside an inner edge of each of the first and second power-transmission antennas, and supplied with power wirelessly so as to generate respective magnetic fields that are in the same direction,
if the axis of the power-reception coil is parallel with a plane that is perpendicular to the axis of the circular ring of each of the first and second power-transmission antennas,
   the first and second power-transmission antennas are arranged coaxially, moved so that the capsule endoscope comes to be located in a region between the respective circular rings bounded by the inner edges and the outer edges of the first and second power-transmission antennas, and supplied with power wirelessly so as to generate respective magnetic fields that are in opposite directions, and
if the axis of the power-reception coil is inclined with respect to a plane that is perpendicular to the axis of the circular ring of each of the first and second power-transmission antennas,
   the first and second power-transmission antennas are deviated from each other so that their central holes overlap with each other, moved so that the capsule endoscope comes to be located in a region of an overlap of the circular rings of the first and second power-transmission antennas, and supplied with power wirelessly so as to generate respective magnetic fields that are in opposite directions, or
   the first and second power-transmission antennas are deviated from each other so that their central holes do not overlap with each other, moved so that the capsule endoscope comes to be located in a region of an overlap of the circular rings of the first and second power-transmission antennas, and supplied with power wirelessly so as to generate respective magnetic fields that are in the same direction.

According to the above mode, the power supply efficiency can be increased further because each of the first power-transmission antenna and the second power-transmission antenna generates magnetic field lines that are in the same direction as the axis of the power-reception coil.

### Advantages of the Invention

According to the capsule endoscope of the invention, efficient power reception is enabled because the magnetic body which is disposed adjoining the inner circumferential surface of the power-reception coil increases the density of a magnetic flux that interlinks with the power-reception coil. With this measure, other members that do not lower the power reception efficiency through generation of an eddy current, such as a non-metal liquid chemical supply tank or microhand device, can be disposed in a wide space secured inside the power-reception coil. As a result, utilizing the space inside the capsule effectively, the capsule endoscope, through small in size, is given a high-grade function of administering a liquid chemical or sampling tissue using an external control signal without lowering in power reception efficiency.

According to the capsule endoscope examination instrument of the invention, because of the employment of the power-transmission antenna or antennas capable of forming a divergent magnetic field directed in all directions, the unit to detect a power reception state and communicating it by a wireless communication or the detector to detect a position or a posture of the capsule, the power-transmission antenna or antennas can be set so that the received power becomes larger than or equal to a prescribed value wherever the capsule endoscope is located. This makes is possible to perform an endoscope examination stably by securing necessary received power. Furthermore, even if the posture of the capsule endoscope is changed suddenly by a vermicular movement of a tubular organ, the amount of received power can be recovered in a short time by moving the power-transmission antenna or antennas quickly. Thus, no large-capacity electricity storage to be used at the time of deterioration of the power reception state is necessary, whereby the capsule endoscope can be miniaturized.

With the above advantages, the capsule endoscope according to the invention is easy to swallow in spite of its multifunctionality because it is kept small, does not cause interruption of an examination because it is supplied with power stably, and can make the physical and mental loads on a subject of an examination even lighter than in conventional cases.

### BRIEF DESCRIPTION OF THE DRAWINGS

FIG. 1 A schematic sectional view of a capsule endoscope according to an embodiment of the present invention.

FIG. 2 A schematic sectional view of a capsule endoscope according to another embodiment of the invention.

FIGS. 3A to 3C Schematic views illustrating how a microhand device shown in FIG. 2 operates.

FIGS. 4A and 4B Schematic sectional views of a capsule endoscope according to still another embodiment of the invention.

FIGS. 5A to 5E Schematic views illustrating an assembling procedure of a power-reception coil shown in FIGS. 4A and 4B.

FIGS. 6A to 6C Schematic sectional views of a capsule endoscope according to still another embodiment of the invention.

FIGS. 7A to 7F Schematic views illustrating an assembling procedure of a power-reception coil shown in FIGS. 6A to 6C.

FIG. 8 A block diagram showing a system configuration of a capsule endoscope according to an embodiment of the invention.

FIG. 9 A circuit diagram showing an example configuration of a rectification/voltage conversion unit shown in FIG. 8.

FIG. 10 A time chart illustrating an example capsule endoscope examining method according to the invention.

FIG. 11 A diagram showing the overall configuration of a capsule endoscope examination instrument according to an embodiment of the invention.

FIG. 12 A block diagram showing the system configuration of a capsule endoscope examination instrument according to an embodiment of the invention.

FIG. 13 A block diagram showing the system configuration of a capsule endoscope examination instrument according to another embodiment of the invention.

FIGS. 14A and 14B Schematic views of a power-transmission antenna according to an embodiment of the invention.

FIG. 15 A diagram showing a magnetic field generated by a power-transmission antenna in vectors.

FIG. 16 A diagram showing strengths of the magnetic field generated by the power-transmission antenna as contour lines.

FIG. 17 A diagram showing magnetic field lines of the magnetic field generated by the power-transmission antenna.

FIG. 18 A diagram in which isoclinic lines are drawn on the basis of inclinations of magnetic field lines of the magnetic field generated by the power-transmission antenna and the space is thereby classified into regions.

FIGS. 19A to 19D Schematic views each of which shows a relationship between a posture of the capsule endoscope and a position of the power-transmission antenna.

FIG. 20 A flowchart illustrating a method for setting the power-transmission antenna at a most appropriate position.

FIGS. 21A and 21B Diagrams illustrating one mode in which the power-transmission antenna is moved by the capsule endoscope examination instrument according to the invention so that the received power is maximized.

FIGS. 22A and 22B Diagrams illustrating another mode in which the power-transmission antenna is moved by the capsule endoscope examination instrument according to the invention so that the received power is maximized.

FIGS. 23A and 23B Diagrams illustrating a still another mode in which the power-transmission antenna is moved by the capsule endoscope examination instrument according to the invention so that the received power is maximized.

FIGS. 24A and 24B Diagrams illustrating a still another mode in which the power-transmission antenna is moved by the capsule endoscope examination instrument according to the invention so that the received power is maximized.

FIG. 25 A schematic diagram illustrating one mode in which the power-transmission antennas are moved by a capsule endoscope examination instrument according to the invention that uses two power-transmission antennas so that the received power is maximized.

FIG. 26 A schematic diagram illustrating another mode in which the power-transmission antennas are moved by the capsule endoscope examination instrument according to the invention that uses the two power-transmission antennas so that the received power is maximized.

FIGS. 27A and 27B Schematic diagrams illustrating a still another mode in which the power-transmission antennas are moved by the capsule endoscope examination instrument according to the invention that uses the two power-transmission antennas so that the received power is maximized.

FIGS. 28A and 28B Schematic diagrams illustrating a still another mode in which the power-transmission antennas are moved by the capsule endoscope examination instrument according to the invention that uses the two power-transmission antennas so that the received power is maximized.

FIG. 29 A graph showing a relationship between the magnitude of an induced electromotive force and the thickness of a cylindrical magnetic body that is attached to a power-reception coil.

FIG. 30 A graph showing a relationship between the magnitude of an induced electromotive force and the relative permeability of the cylindrical magnetic body that is attached to the power-reception coil.

### MODES FOR CARRYING OUT THE INVENTION

Capsule endoscopes according to embodiments of the present invention will be hereinafter described with reference to the drawings. The drawings are schematic ones, and the shapes, the ratios between dimensions, etc. in the drawings are not exactly the same as real ones. The same members etc. are given the same symbol and descriptions therefor may be omitted.

### [Capsule Endoscope]

The configuration of a capsule endoscope will be described below in a specific manner.

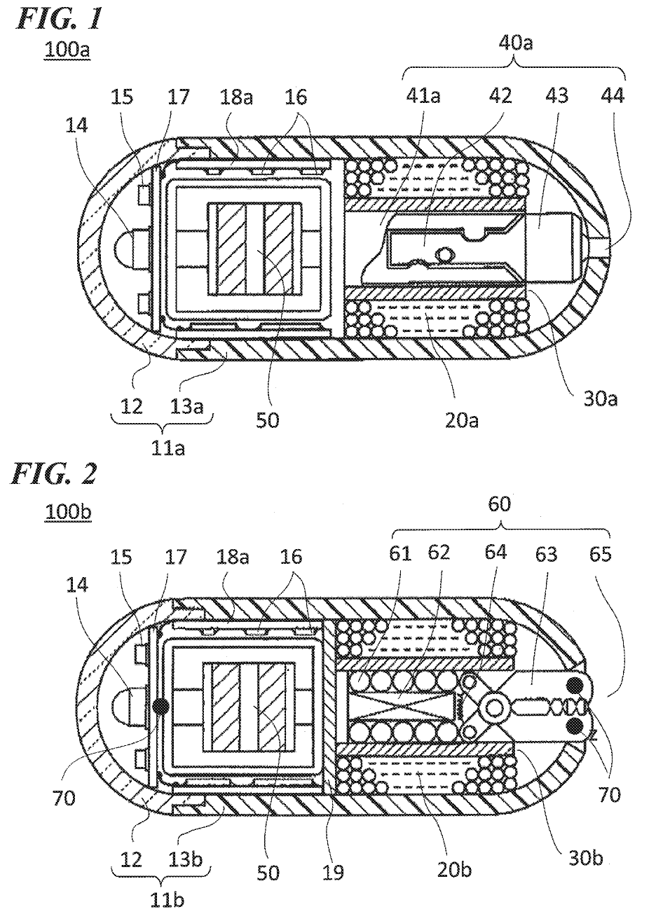

FIG. 1 is a schematic sectional view of a capsule endoscope according to an embodiment of the invention. This capsule endoscope 100a is equipped with a capsule 11a which is formed by connecting a hemispherical tip cover 12 formed by a transparent member and a generally cylindrical capsule barrel 13a having a hemispherical end portion. To enable shooting of the inside of tubular organs, an electronic circuit board 17 which is mounted with a camera 14 and illumination devices 15 is disposed inside the transparent tip cover 12. Inside the generally cylindrical capsule barrel 13a, a self-propulsion drive device 50 and a power-reception coil 20a are disposed in series behind the electronic circuit board 17. A magnetic body 30a is disposed adjoining the inner circumferential surface of the power-reception coil 20a. An electronic circuit board(s) 18a which is mounted with semiconductor devices 16 is disposed in the gap between the generally cylindrical capsule barrel 13a and the self-propulsion drive device 50. A liquid chemical supply device 40a is disposed in the inside space of the magnetic body 30a.

The capsule endoscope 100a can go into tubular organs such as the digestive organs, move inside the tubular organs using the self-propulsion drive device 50, shoot the inside of the tubular organs using the camera 14, and administer a liquid chemical using the liquid chemical supply device 40a.

The capsule 11a is composed by connecting the hemispherical tip cover 12 formed by a transparent member and the generally cylindrical capsule barrel 13a having the hemispherical end portion and has a hermetic structure prevent the camera 14, the illumination device 15 and the semiconductor devices 16 from coming into contact with liquid. It is preferable that the outer size and the length of the capsule 11a be about 9 to 12 mm and about 20 to 30 mm, respectively. The capsule 11a is difficult to ingest if its size or length exceeds this range, and it is difficult to incorporate the necessary members in the capsule 11a if its size or length is smaller than this range.

The camera 14 is mounted on the electronic circuit board 17 together with the illumination devices 15 so that it is adjacent to the tip cover 12 and the tip of its lens is directed to the tip cover 12 and that it can thus shoot the outside through the transparent tip cover 12. Composed of the lens and a solid-state imaging device, the camera 14 can convert an image formed by performing photoelectric conversion on it. No particular limitations are imposed on the type of the solid-state imaging device; specific examples are a CCD (charge-coupled device) image sensor and a CMOS (complementary metal-oxide-semiconductor) image sensor.

The illumination devices 15 are used for illuminating a shooting target of the camera 14 to make it brighter. It is preferable that the illumination devices 15 be of a power saving type; more specifically, the use of white light-emitting diodes is preferable.

The electronic circuit board(s) 18a is mounted with the semiconductor devices 16 having such functions as signal processing, wireless communication, and power control. The electronic circuit board(s) 18a may be a common circuit board(s) made of an epoxy resin or be made of a flexible material and wound into a tubular shape. Alternatively, the electronic circuit board(s) 18a may be divided into plural boards or, conversely, be integral with the electronic circuit board 17. There are no particular limitations on the installation place of the electronic circuit board(s) 18a; for example, it may be disposed inside the power-reception coil 20a. The amounts of conductive materials used in the electronic circuit board(s) 18a are very small and hence no eddy current large enough to affect the power reception efficiency is generated there.

In the capsule endoscope 100a, the power-reception coil 20a is a cylindrical coil that is a winding of a coated wire. For example, the power-reception coil 20a may be a hollow coil of 9 mm in outer diameter (smaller than the inner diameter of the capsule 11a) and 6 mm in inner diameter that is formed by winding, into a cylindrical shape, a coated wire of 0.3 mm in outer diameter and 2 m in length that has a copper core wire of 0.1 mm in diameter and a thick insulating coating layer. The coated wire may be wound either clockwise or counterclockwise and may have some disorder in winding form. To avoid cancellation between magnetic field components, it is preferable wind the coated wire entirely in the same direction and in order.

In the capsule endoscope 100a, the magnetic body 30a may be disposed inside the power-reception coil 20a. No particular limitations are imposed on the magnetic body 30a; it is preferable that the magnetic body 30a be a member that is shaped into a cylindrical form and made of a ferromagnetic material such as ferrite, cobalt, iron, iron oxide, chromium oxide, or nickel, and it is particularly preferable that the magnetic body 30a be a member produced by curling, by one turn, a resin sheet of 0.1 to 0.5 mm in thickness containing ferrite. When produced by curling a ferrite resin sheet, the magnetic body 30a is high in magnetic flux density at the inner surface of the power-reception coil 20a. Thus, when the magnetic body 30a is used, the received power (power value) is 15 times (thickness 0.1 mm), 21 times (0.2 mm), and 25 times (0.5 mm) as large as that of a case that no magnetic body is used. Where the magnetic body 30a is formed using a thin ferrite resin sheet, a wide space in which various members can be placed and used effectively is formed inside the power-reception coil 20a.

In the capsule endoscope 100a, the space inside the power-reception coil 20a can be utilized effectively by disposing a non-metal (resin or ceramic) liquid chemical tank 41a there. Since the liquid chemical tank 41a is made of a non-metal, no eddy current to cause reduction in power reception efficiency is generated there. The liquid chemical supply device 40a is composed of the liquid chemical tank 41a, a liquid chemical suction pipe 42 which is inserted in the liquid chemical tank 41a, an electromotive valve or pump 43 which is driven by power received by the power-reception coil 20a, and a liquid chemical emission opening 44 which is formed at one end of the capsule 11a. Since the electromotive valve or pump 43 can be driven by a control signal that is supplied externally, a liquid chemical can be administered at a desired time and place.

The capsule endoscope 100a makes it possible to examine a wide range in a short time because it is equipped with the self-propulsion drive device 50 and hence can move inside tubular organs and shoot the inside of them. There are no particular limitations on the self-propulsion drive device 50. For example, an actuator having an electromagnet and a permanent magnet can be used in which electric power obtained by the power-reception coil 20a is used as a motive power source, that is, a magnetic field is generated by causing a current to flow through a coil (to serve as the electromagnet) wound on a cylindrical case and a rod magnet (permanent magnet) placed inside the case is thereby caused to collide with a wall surface strongly that is located on the destination side in its movement direction to obtain propulsion (the rod magnet is returned slowly by causing a weak reverse current through the coil). However, since the self-propulsion drive device 50 includes the magnetic members and the conductive member (coil), it is not preferable that the self-propulsion drive device 50 is disposed inside the power-reception coil 20a. Not to impair the magnetic flux density enhancing effect of the magnetic body 30a which is disposed adjoining the inner circumferential surface of the power-reception coil 20a, it is preferable that the self-propulsion drive device 50 be disposed in series to the power-reception coil 20a along the axial direction of the capsule 11a so that the self-propulsion drive device 50 (more specifically, at least the permanent magnet) does not enter the inside of the power-reception coil 20a.

FIG. 2 is a schematic sectional view of a capsule endoscope according to another embodiment of the invention. This capsule endoscope 100b is equipped with a capsule 11b which is formed by connecting a hemispherical tip cover 12 formed by a transparent member and a generally cylindrical capsule barrel 13b having a hemispherical end portion. The capsule 11b is separated into two rooms by a partition wall 19, and the room on the side of the transparent tip cover 12 is sealed. To enable shooting of the inside of tubular organs, an electronic circuit board 17 which is mounted with a camera 14 and illumination devices 15 is disposed inside the transparent tip cover 12. On the other hand, in the generally cylindrical capsule barrel 13b, a self-propulsion drive device 50, a power-reception coil 20b (behind the self-propulsion drive device 50), and a magnetic body 30b (adjoining the inner circumferential surface of the power-reception coil 20b). An electronic circuit board(s) 18a which is mounted with semiconductor devices 16 is disposed in the gap between the generally cylindrical capsule barrel 13b and the self-propulsion drive device 50. A microhand device 60 is disposed in the inside space of the magnetic body 30b.

The microhand device 60 is composed of a resin shape-memorized spring 61 which is memorized with an elongated shape at a high temperature and housed in a compressed state at low temperatures, a ceramic heater 62 for heating the resin shape-memorized spring 61, non-metal (resin or ceramic) scissors 63 which are attached to the tip of the shape-memorized spring 61, and a resin spring 64 for opening the scissors 63. The microhand device 60 can be driven by power received by the power-reception coil 20b. Since all of the components of the microhand device 60 are made of non-metal materials, the microhand device 60 does not lower the power reception efficiency even if it is disposed in the inside space of the power-reception coil 20b. Thus, the capsule 11b can be miniaturized by utilizing the inside space of the power-reception coil 20b effectively.

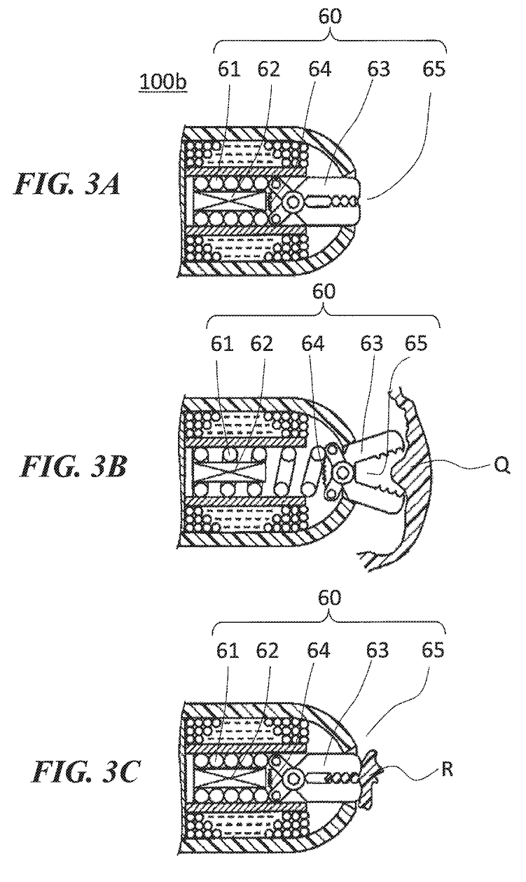

How the microhand device 60 operates will be described with reference to FIGS. 3A to 3C. As shown in FIG. 3B, when the ceramic heater 62 is energized, the shape-memorized spring 61 is expanded, whereupon the scissors 63 project from an opening 65 formed at one end of the capsule 11b, are opened by the contraction force of the spring 64, and come into contact with the inner wall of a tubular organ Q. As shown in FIG. 3C, when the energization is stopped, the shape-memorized spring 61 cools and pulls back the scissors 63. During that course, the scissors 63 are closed as they come to be confined in the opening 65 which is formed at the one end of the capsule 11b. When the scissors 63 are closed, they can bite off part of the tubular organ Q and take it as a sample R.

Since the microhand device 60 employed in the invention uses sufficient power that is received efficiently and has a simple structure that the scissors 63 are opened and closed by energizing and deenergizing to the ceramic heater 62, respectively, the microhand device 60 has features that they are small and hard to fail. Furthermore, capable of being driven by an external control signal, the microhand device 60 can take a sample at a desired time and place.

The capsule endoscope according to the invention may be equipped with X-ray markers for detection of a position and a posture at at least two locations in its longitudinal direction. For example, as shown in FIG. 2, a total of three X-ray markers 70 may be provided on the back surface of the electronic circuit board 17 and at the tips of the scissors 63 of the microhand device 60. Providing the three X-ray markers 70 makes it possible to determine whether the capsule endoscope 100b is directed forward or backward. There are no particular limitations on the material of the X-ray markers 70 except that it should not transmit X-rays easily; preferable examples are gold, platinum, and a tantalum alloy.

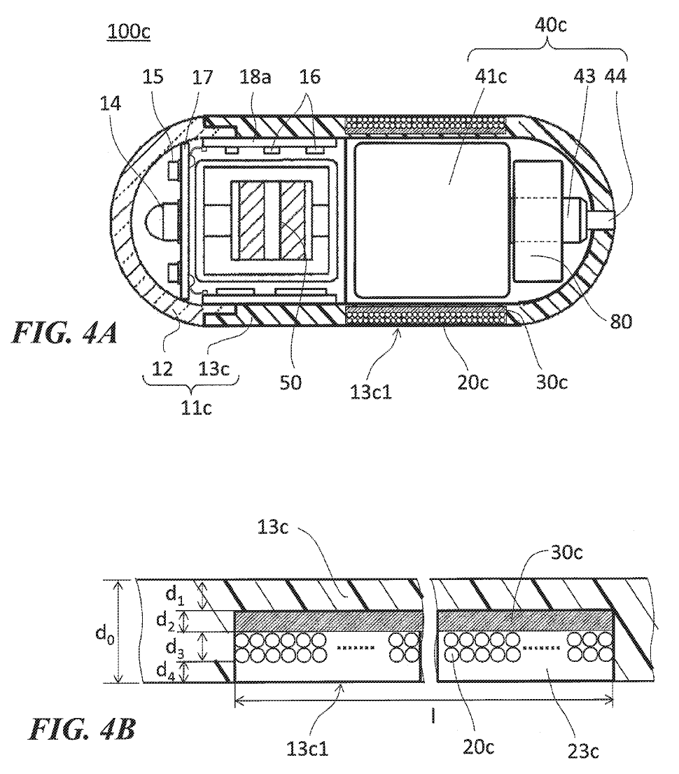

FIG. 4A is a schematic sectional view of a capsule endoscope according to still another embodiment of the invention. This capsule endoscope 100c is equipped with a capsule 11c which is formed by connecting a hemispherical tip cover 12 formed by a transparent member and a generally cylindrical capsule barrel 13c having a hemispherical end portion. To enable shooting of the inside of tubular organs, an electronic circuit board 17 which is mounted with a camera 14 and illumination devices 15 is disposed inside the transparent tip cover 12.

In the embodiment, a power-reception coil 20c and a magnetic body 30c are provided within the wall thickness of the generally cylindrical capsule barrel 13c. With this structure, the inside space of the power-reception coil 20c can be made wider and utilized more efficiently than in the capsule endoscope 100a shown in FIG. 1.

A self-propulsion drive device 50, a liquid chemical supply device 40c, and a capacitor 80 are disposed in this order inside the capsule barrel 13c. The self-propulsion drive device 50 is composed of high permeability members and conductive members.

The capacitor 80 is equipped with conductive electrode members. It is therefore not preferable to dispose each of the self-propulsion drive device 50 and the capacitor 80 inside the power-reception coil 20c. Not to impair the magnetic flux density enhancing effect of the magnetic body 30c which is disposed adjoining the inner circumferential surface of the power-reception coil 20c, it is preferable that each of the self-propulsion drive device 50 and the capacitor 80 be disposed in series to the power-reception coil 20c. Since the liquid chemical supply device 40c is composed of non-metal members and hence do not affect a magnetic flux, it is disposed inside the power-reception coil 20c. An electronic circuit board(s) 18a which is mounted with semiconductor devices 16 is disposed in the gap between the generally cylindrical capsule barrel 13c and the self-propulsion drive device 50.

FIG. 4B is an enlarged sectional view of the power-reception coil 20c. Part of the outer circumferential surface of the cylindrical portion of the capsule barrel 13c is formed with a ring-shaped recess 13c1. The magnetic body 30c is disposed at the bottom of the recess 13c1, and the power-reception coil 20c is disposed on the outer circumferential surface of the magnetic body 30c and covered with a coating layer 23c. In this manner, the power-reception coil 20c and the magnetic body 30c are provided within the wall thickness of the capsule barrel 13c.

There are no particular limitations on the dimensions of the individual portions relating to the power-reception coil 20c. For example, preferable modes are as follows.

To secure necessary mechanical strength, it is preferable that the thickness do of the capsule barrel 13c be 0.8 to 1.0 mm and the thickness d₁ of wall of the recess 13c1 be larger than or equal to 0.2 mm. It is preferable that the magnetic body 30c be a cylindrical magnetic body of 0.2 to 0.3 mm in thickness d₂ formed by curling a resin sheet (e.g., ferrite resin sheet) of 100 to 130 in relative permeability and 0.2 to 0.3 mm in thickness containing a ferroelectric material. It is preferable that the power-reception coil 20c be a cylindrical coil of 0.24 to 0.3 mm in thickness d₃ and 4 to 6.5 mm in coil length I formed by winding a coated wire of 0.10 to 0.15 mm in outer diameter around the outer circumferential surface of the magnetic body 30c in two layers. It is preferable that the outer circumferential surface of the power-reception coil 20c be coated with a coating layer 23c made of a resin or the like. Although there are no particular limitations on the thickness d₄ of the coating layer 23c, it is preferable that, for example, the thickness d₄ be set so that no steps are formed in the capsule surface to reduce frictional resistance and allow a subject to swallow the capsule endoscope 100c more easily.

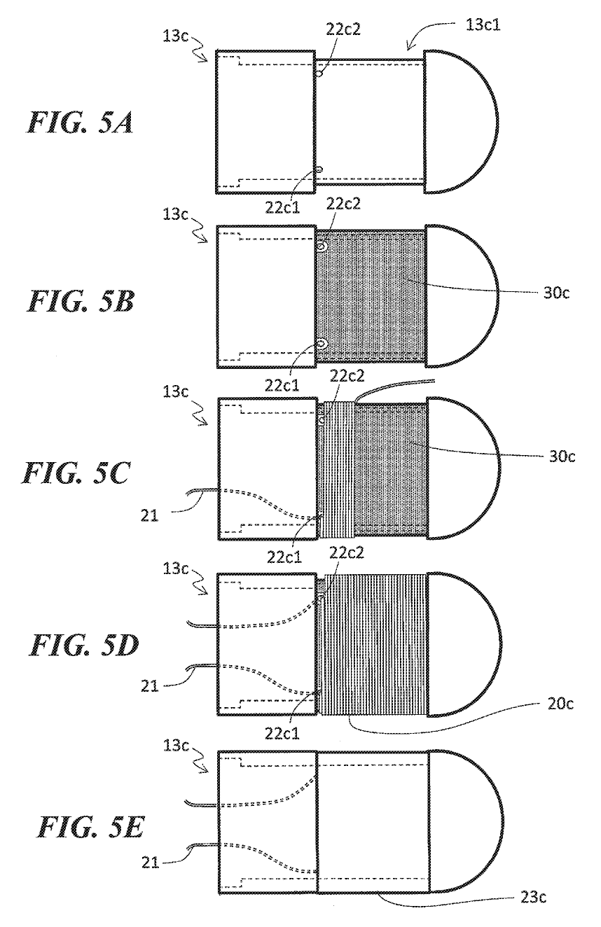

FIGS. 5A to 5E are schematic views illustrating an assembling procedure of the power-reception coil 20c. As shown in FIG. 5A, the outer circumferential surface of the generally cylindrical capsule barrel 13c having the hemispherical end portion is formed with the ring-shaped recess 13c1. Through-holes 22c1 and 22c2 through which to insert a coated wire penetrate through the wall of the recess 13c1. As shown in FIG. 5B, a cylindrical magnetic body 30c is formed by winding, around the wall of the recess 13c1, by one turn, a resin sheet that contains a ferromagnetic material and is formed with cuts at such positions as to be registered with the respective through-holes 22c1 and 22c2. Then, as shown in FIG. 5C, one end portion of a coated wire 21 is introduced to outside the capsule barrel 13c through the through-hole 22c1 from inside the capsule barrel 13c and wound around the outer circumference of the magnetic body 30c. Then, as shown in FIG. 5D, after a power-reception coil 20c is formed by winding the coated wire 21, an end portion of the remaining part of the coated wire 21 is returned to inside the capsule barrel 13c through the through-hole 22c2. Finally, as shown in FIG. 5E, the outer circumferential surface of the power-reception coil 20c is covered with a resin or the like to form a coating layer 23c.

Capable of having a wider space than the capsule endoscope 100a which is equipped with the inside power-reception coil 20a, the capsule endoscope 100c of the above mode which is equipped with the outside power-reception coil 20c can be equipped with, for example, a larger liquid chemical tank.

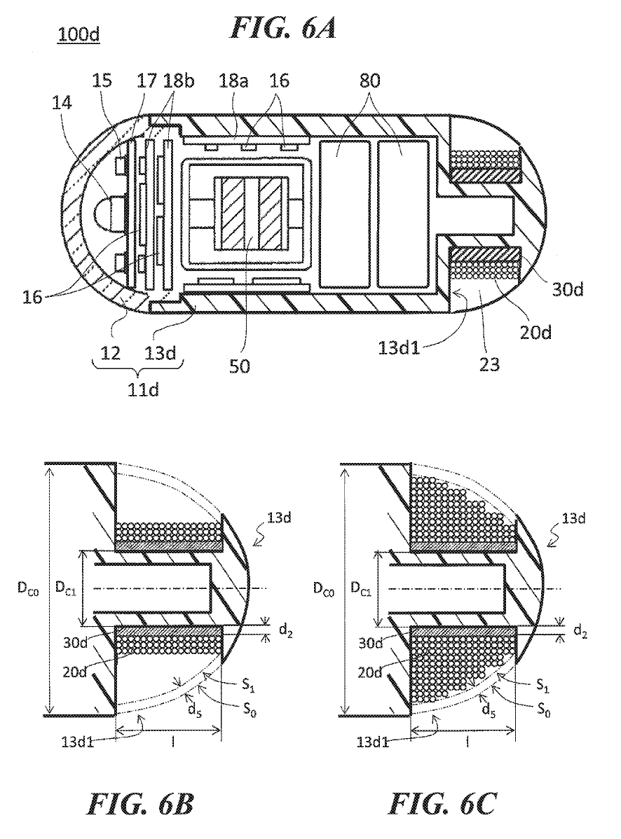

FIG. 6A is a schematic sectional view of a capsule endoscope according to a further embodiment of the invention. This capsule endoscope 100d is equipped with a capsule 11d which is formed by connecting a hemispherical tip cover 12 formed by a transparent member and a generally cylindrical capsule barrel 13d having a hemispherical end portion. To enable shooting of the inside of tubular organs, an electronic circuit board 17 which is mounted with a camera 14 and illumination devices 15 is disposed inside the transparent tip cover 12.

In the embodiment, a power-reception coil 20d and a magnetic body 30d are disposed in the hemispherical end portion of the capsule barrel 13d. With this configuration, it is possible to employ, as a place to dispose the power-reception coil 20d and the magnetic body 30d, the hemispherical end portion whose free space is difficult to use and to dispose high permeability members and conductive members in a desired manner in the inside space of the cylindrical portion of the capsule barrel 13d.

Inside the cylindrical portion of the capsule barrel 13d, an electronic circuit board(s) 18a which is mounted with semiconductor devices 16 is disposed in the gap between a self-propulsion drive device 50 and the capsule barrel 13d and two electronic circuit boards 18b which are mounted with semiconductor devices 16 are disposed behind the electronic circuit board 17 which is mounted with the camera 14 and the illumination devices 15. Two large capacity capacitors 80 are disposed behind the self-propulsion drive device 50 to enable supply of more electricity.

FIG. 6B is an enlarged sectional view of the power-reception coil 20d. The outer circumferential surface of the hemispherical end portion of the capsule barrel 13d is formed with a ring-shaped recess 13d1. The magnetic body 30d is disposed at the bottom of the recess 13d1, and the power-reception coil 20d is disposed on the outer circumferential surface of the magnetic body 30d and covered with a coating layer 23d. In this manner, the power-reception coil 20d and the magnetic body 30d are housed in the spherical end portion of the capsule barrel 13d.

There are no particular limitations on the dimensions of the individual portions relating to the power-reception coil 20d. For example, preferable modes are as follows.

There are no particular limitations on the diameter (coil diameter) D_{C1} of the cylindrical constricted portion of the ring-shaped recess 13d1 of the capsule barrel 13d. However, since the length I of the ring-shaped recess 13c1 (coil length) becomes shorter as the diameter D_{C1} increases, it is preferable that the diameter D_{C1} be about ½ of the outer diameter D_{C0} of the cylindrical portion of the capsule barrel 13c. For example, the coil length I may be made 4 mm when the coil diameter D_{C1} is set at 5 mm for D_{C0} being equal to 11 mm.

It is preferable that the magnetic body 30d be a cylindrical magnetic body of 0.1 to 0.5 mm in thickness d₂ formed by curling a resin sheet (e.g., ferrite resin sheet) of 100 to 130 in relative permeability and 0.1 to 0.5 mm in thickness containing a ferroelectric material. It is preferable that the power-reception coil 20d be a cylindrical coil of 3 to 4 mm in coil length I formed by winding a coated wire of 0.10 to 0.15 mm in outer diameter around the outer circumferential surface of the magnetic body 30d in three or more layers.

As shown in FIG. 6C, the coated wire may be wound in such a manner that the number of superimposition layers increases as the position comes closer to the cylindrical portion of the capsule barrel 13d and decreases as the position comes closer to the end of the capsule barrel 13d, in other words, the width of the coil cross section decreases as the position comes closer to the end of the capsule barrel 13d. However, to make it possible to form a coating layer 23d by injection molding, the number of superimposition layers is restricted so that the distance d₅ between the imaginary surface S₀ of the partially recessed hemispherical portion of the capsule barrel 13d and the surface S₁ of the power-reception coil 20c becomes longer than or equal to 4 mm.

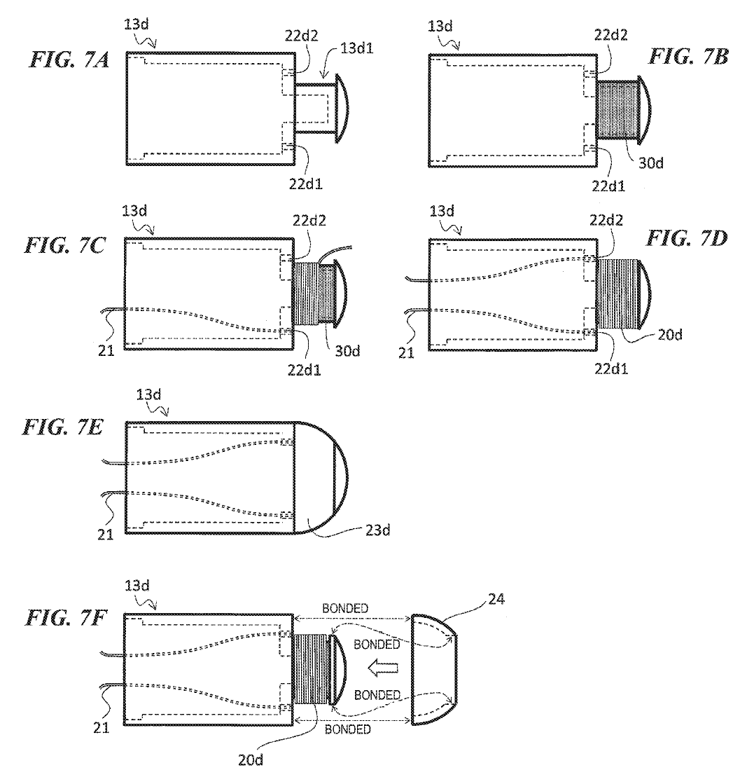

FIGS. 7A to 7F are schematic views illustrating an assembling procedure of the power-reception coil 20d. As shown in FIG. 7A, the end portion, where to form a hemispherical structure when completed, of the capsule barrel 13d is formed with the ring-shaped recess 13d1. Through-holes 22d1 and 22d2 through which to insert a coated wire penetrate through one side wall of the recess 13d1. As shown in FIG. 7B, a cylindrical magnetic body 30d is formed by winding, around the core wall of the recess 13d1, by one turn, a resin sheet that contains a ferromagnetic material. Then, as shown in FIG. 7C, one end portion of a coated wire 21 is introduced to outside the capsule barrel 13d through the through-hole 22d1 from inside the capsule barrel 13d and wound around the outer circumference of the magnetic body 30d. Then, as shown in FIG. 7D, after a power-reception coil 20d is formed by winding the coated wire 21, an end portion of the remaining part of the coated wire 21 is returned to inside the capsule barrel 13d through the through-hole 22d2. Finally, as shown in FIG. 7E, the outer circumferential surface of the power-reception coil 20d is covered with a resin by injection molding to form a coating layer 23d whose surface assumes part of a hemisphere. In terms of unitization with melting, it is preferable that the resin material used for the injection molding be the same as or similar to the material of the capsule barrel 13d.

Alternatively, as shown in FIG. 7F, a method for covering the power-reception coil 20d may be that a resin cap 24 whose surface assumes part of a hemisphere and which conforms to the end portion of the capsule barrel 13d and has a top opening is bonded or welded to the capsule barrel 13d. This method can lower the manufacturing cost because it can be performed more easily than the unitization by injection molding. The material of the cap 24 need not always be the same as or similar to that of the capsule barrel 13d; the thickness of the cap 24 can be reduced by using a stronger resin material. The power reception efficiency can be increased by increasing the diameter of the power-reception coil 20d by a reduction of the thickness of the cap 24.

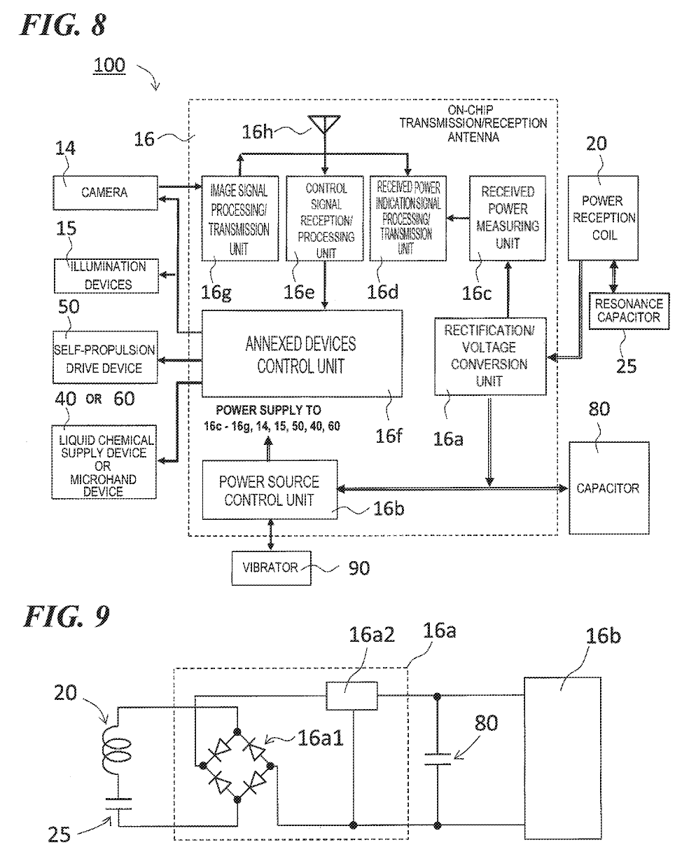

Next, an example system configuration of a capsule endoscope 100 according to the invention will be described with reference to FIG. 8. A semiconductor device 16 can be equipped with a rectification/voltage conversion unit 16a, a power source control unit 16b, a received power measuring unit 16c, a received power indication signal processing/transmission unit 16d, a control signal reception/processing unit 16e, an annexed devices control unit 16f, an image signal processing/transmission unit 16g, and an on-chip transmission/reception antenna 16h. The camera 14, illumination devices 15, the self-propulsion drive device 50, the liquid chemical supply device 40 or the microhand device 60, the power-reception coil 20, a resonance capacitor 25, the capacitor 80, and a vibrator 90 can be connected to the semiconductor device 16. The semiconductor device 16 need not always be of a single chip, and may be divided into plural chips that correspond to respective function blocks.

The rectification/voltage conversion unit 16a converts AC power received by an LC resonance circuit that is a series connection of the power-reception coil 20 and the resonance capacitor 25 into DC power of a prescribed voltage and supplies the latter to the capacitor 80 and the power source control unit 16b. For example, as shown in FIG. 9, equipped with a diode bridge 16a1 and a booster circuit 16a2, the rectification/voltage conversion unit 16a can boost with the booster circuit 16a2 DC power produced by the diode bridge 16a1 through rectification and store resulting DC power in the capacitor 80 temporarily.

The capacitor 80 has a role of suppressing a voltage variation by storing charge. It is preferable that the capacitor 80 be small and of a large capacity; for example, an electric double-layer capacitor is used preferably. The capacitor 80 may be replaced by a storage battery such as a lithium-ion secondary battery.

The power source control unit 16b is equipped with a voltage regulation circuit (linear regulator), a power source protection circuit, a reference voltage circuit, an oscillation circuit, etc., and has roles of stable supply of power, monitoring, shutting-off, reference voltage/clock generation. To generate a highly accurate clock signal wave and carrier wave for wireless communication, it is desirable that the power source control unit 16b be connected to the vibrator 90 such as a quartz vibrator or a ceramic vibrator.

The received power measuring unit 16c can measure the magnitude of AC power received by the power-reception coil 20 in the form of the magnitude of a DC voltage obtained by the rectification/voltage conversion unit 16a through conversion of the AC power.

The received power indication signal processing/reception unit 16d can encode the magnitude of measured received power into a digital signal and send the latter from the transmission/reception antenna 16h.

The control signal reception/processing unit 16e can receive, via the transmission/reception antenna 16h, a control signal transmitted from the outside, decode it, and send a resulting signal to the annexed devices control unit 16f.

The annexed devices control unit 16f can control the camera 14, the illumination devices 15, the self-propulsion drive device 50, the liquid chemical supply device 40, and the microhand device 60.

The image signal processing/transmission unit 16g can perform signal processing on an image signal taken by the camera 14 and send a resulting signal to the outside via the transmission/reception antenna 16h.

### [Capsule Endoscope Examining Method]

In the capsule endoscope 100 according to the invention, the power-reception coil may be supplied with power continuously. Alternatively, as described below, it may be supplied with power intermittently.

For example, where there is concern that continuous operation of the self-propulsion drive device 50 may cause temperature increase in the capsule endoscope 100, the power-reception coil 20 may be supplied with power intermittently in the following manner. During power supply periods (power supply on periods), the capsule endoscope 100 is caused to move on its own by energizing the self-propulsion drive device 50. During non-power-supply periods (power supply off periods), the capsule endoscope 100 is allowed to cool by deenergizing the self-propulsion drive device 50.

Where there is concern that a communication error may occur due to electromagnetic noise during power supply on periods, the power-reception coil 20 may be supplied with power intermittently in the following manner During power supply off periods, the transmission/reception units 16d, 16e, and 16g are allowed to communicate with the outside. During power supply on periods, wireless communications are suspended.

A start and an end of a power supply off period can be detected by a power measurement unit to measure the magnitude of power received by the power-reception coil or a time measurement unit that operates in synchronism with supply of power to the power-reception coil 20. More specifically, the received power measuring unit 16c can be provided as a unit to measure the magnitude of received power and the power control unit 16b having a counter circuit for counting clocks can be provided as a time measurement unit that operates in synchronism with supply of power to the power-reception coil 20. Using these units, the capsule endoscope 100 can be controlled in synchronism with intermittent supply of power by detecting a start and an end of a power supply off period.

An example manner of intermittent supply of power will be described below in detail with reference to the related drawings.

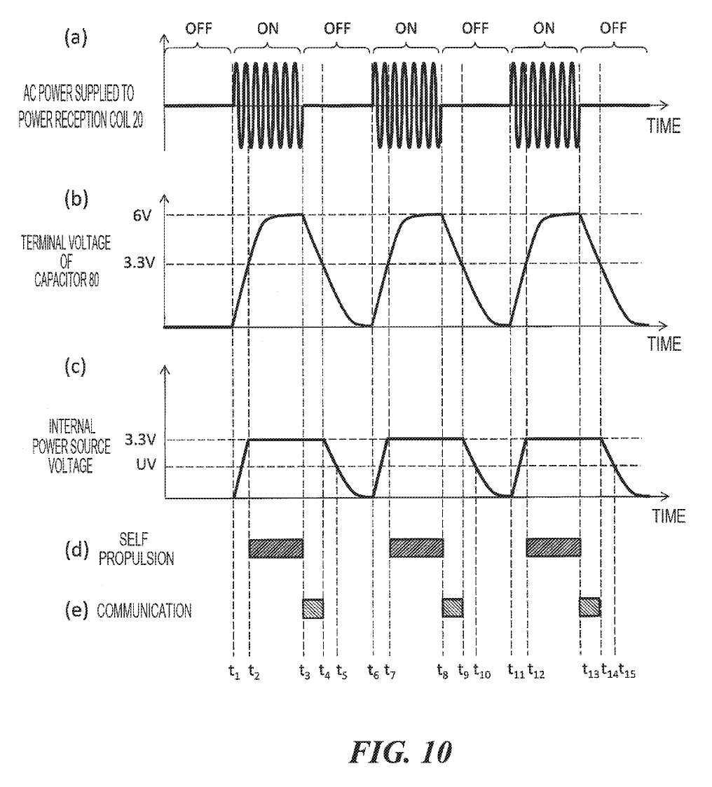

Part (a) of FIG. 10 shows an on/off waveform of AC power that is supplied to the power-reception coil 20 intermittently. A fundamental frequency of AC power is selected from a frequency range of 50 to 500 kHz, and a power supply on/off switching cycle of intermittent supply of power is set longer than the fundamental frequency. A power supply on period being shorter than 100 ms is not preferable because the operation frequency of the self-propulsion drive device 50 is several tens of hertz and hence it is difficult to enable normal operation of the self-propulsion drive device 50 with such a power supply on period. On the other hand, if the power supply off period is as long as several seconds, supply of power to the semiconductor device 16 is stopped to reset the circuit of the semiconductor device 16. For these reasons, it is preferable that the power supply on/off switching cycle of intermittent supply of power be set at 100 to 1,000 ms. The duty ratio may be set as appropriate according to the situation. For example, by setting each of the power supply on period and the power supply off period at 100 ms, the capsule endoscope 100 can send a moving image (five frames per sec) taken by the camera 14 while moving on its own.

Part (b) of FIG. 10 shows a waveform of the terminal voltage of the capacitor 80. Part (c) of FIG. 10 shows a waveform of an internal power source voltage of the semiconductor device 16 produced by lowering the terminal voltage of the capacitor 80 by the linear regulator. Part (d) of FIG. 10 shows periods when the capsule endoscope 100 is caused to move on its own. Part (e) of FIG. 10 shows periods when a communication with the outside is made.

Supply of power is turned on at time t₁. Received AC power is rectified by the diode bridge 16aa and boosted to 6 V by the booster circuit 16ab, and a resulting voltage is supplied to the capacitor 80. Although the terminal voltage of the capacitor 80 increases in proportion to the amount of charge stored therein, the internal power source voltage is clamped at 3.3 V by the linear regulator (at time t₂). On the other hand, the terminal voltage of the capacitor 80 is saturated after being increased to 6 V which is equal to the output voltage 6 V of the booster circuit 16ab. At time t₃, the supply of power is turned off and the terminal voltage of the capacitor 80 starts to decrease. However, since the internal power source voltage is clamped at 3.3 V by the linear regulator, it is kept at 3.3 V until time t₄. When the terminal voltage of the capacitor 80 has become lower than 3.3 V, the internal power source voltage also starts to decrease and reaches, at time t₅, a lower limit voltage UV under which the semiconductor device 16 is rendered non-operational. However, if the circuit is not shut down, the internal power source voltage continues to decrease. At time t₆, supply of power is turned on again, whereupon charging of the capacitor 80 is started. The same operation as described above is repeated thereafter.

The internal power source voltage is kept at 3.3 V from time t₂ to time t₄. For example, operations are possible that from time t₂ to time t₃ the capsule endoscope 100 is allowed to move on its own by supplying power to the self-propulsion drive device 50 while charging the capacitor 80, and that from time t₃ to time t₄ the inside of the digestive tract is shot and an image signal is transmitted to the outside by a wireless communication by supplying power stored in the capacitor 80 to the camera 14, the illumination units 15, and the image signal processing/transmission unit 16g. Control signals can be sent and received in the period of time t₃ to time t₄.

From the viewpoint of the stability of circuit operations, it is desirable to back up the power source by providing a reverse blocking diode and an auxiliary capacitor (neither shown in FIG. 8) downstream of the capacitor 80 so that the received power measuring function and the clock generation function even after the terminal voltage of the capacitor 80 becomes lower than the allowable lower limit.

### [Capsule Endoscope Examination Instrument]

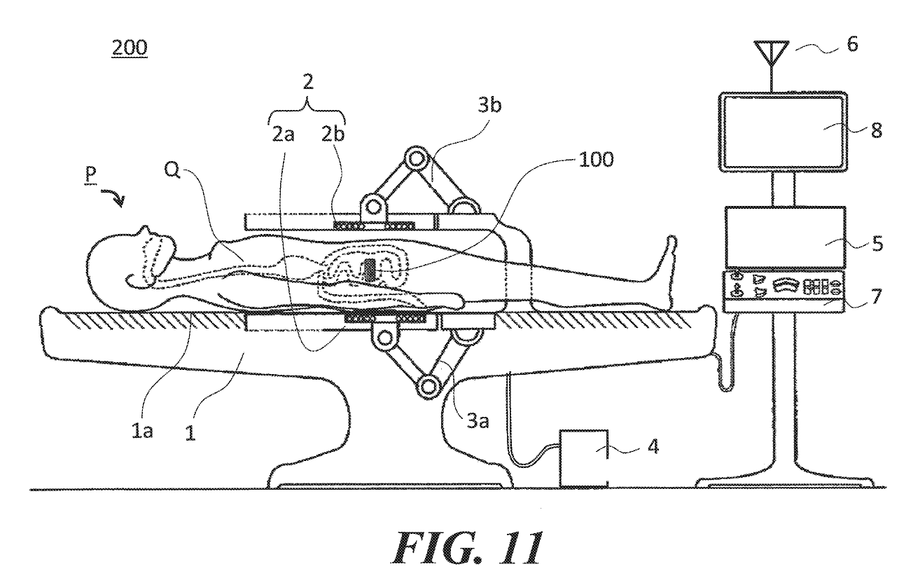

FIG. 11 shows the overall configuration of a capsule endoscope examination instrument 200 according to an embodiment of the invention. The capsule endoscope examination instrument 200 may be equipped with an examination stage 1 on which a subject P is placed, a power-transmission antenna 2a which is disposed under a subject placement part 1a of the examination stage 1 to supply power wirelessly to the capsule endoscope 100, a manipulator 3a for moving the power-transmission antenna 2a, a power-transmission antenna 2b which is disposed over the subject P, a manipulator 3b for moving the power-transmission antenna 2b, an AC power source 4 for supplying AC power to the power-transmission antennas 2a and 2b, a control unit 5, a transmission/reception antenna 6 for wireless communication with the capsule endoscope 100, a manipulation unit 7, and a display unit 8.

In the capsule endoscope examination instrument 200 according to the invention, there are no particular limitations on the power-transmission antennas 2a and 2b; for example, each of them may be a coil formed by winding a conductor in spiral form or a coil formed by winding a conductor in cylindrical form. Since the position and the posture of the capsule endoscope 100 vary from time to time as the digestive tract makes a vermicular movement, it is preferable that the power-transmission antennas 2a and 2b generate a magnetic field capable of accommodating various positions and postures. The power reception efficiency can be maximized by disposing the power-transmission antennas 2a and 2b so that the axis of the cylindrical power-reception coil 20 coincides with the direction of the magnetic field.

Either both or one of the power-transmission antennas 2a and 2b may be provided. Although even one of the power-transmission antennas 2a and 2b enables efficient supply of power if a proper position control is made, a wide region where the strength and the direction of a magnetic field are uniform can be obtained by using both power-transmission antennas 2a and 2b, in which case the supply of power is stabilized.

The manipulators 3a and 3b are capable of moving the power-transmission antennas 2a and 2b to positions where they maximize the power reception efficiency.

The AC power source 4 is capable of supplying AC power to the power-transmission antennas 2a and 2b and is composed of a frequency generator, a DC power source, and an inverter. The frequency of AC power is preferably 50 to 500 kHz, even preferably 100 to 200 kHz, and particularly preferably 150 kHz. Frequencies lower than 50 kHz are not preferable because the overall transmission efficiency is low and it is difficult to obtain a long transmission distance. Frequencies higher than 500 kHz are not preferable because a drive circuit is costly, the driver efficiency is low, and attenuation in a human body is large.

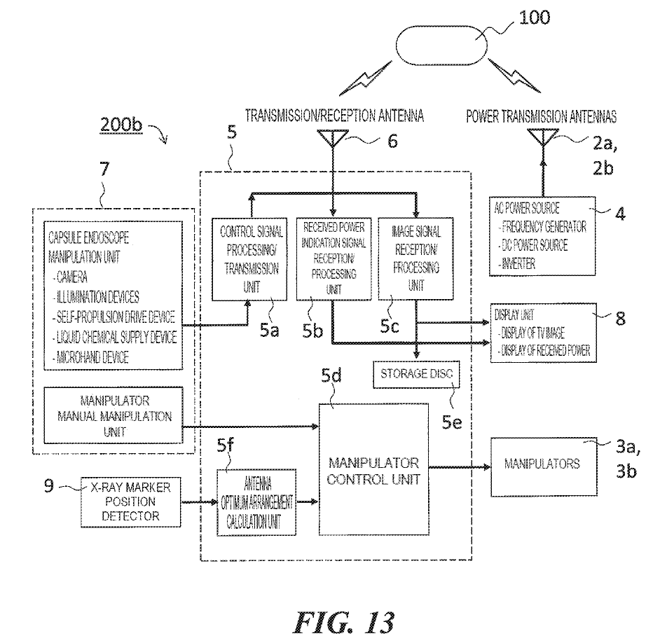

When necessary, the capsule endoscope examination instrument 200 according to the invention may be equipped with an X-ray marker position detector 9 (not shown; see FIG. 13). The X-ray marker position detector 9 can detect a position and a posture of the capsule endoscope 100 by irradiating the abdomen of the subject P with X-rays and analyzing a perspective image including X-ray markers buried in the capsule endoscope 100. In this embodiment, the X-ray markers and the X-ray marker position detector 9 constitute a “detector to detect a position and a posture of a capsule” of the invention. Alternatively, the “detector to detect a position and a posture of a capsule” may also be a unit to detect them using electrical signals that are sent and received by the transmission/reception antenna 16h of the capsule endoscope 100.

### (Example System Configuration 1)

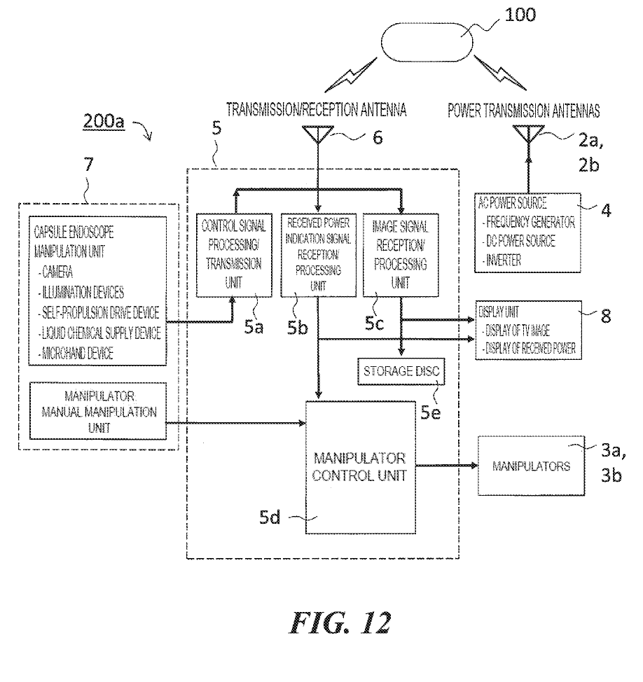

FIG. 12 is a functional block diagram showing the system configuration of one capsule endoscope examination instrument according to the invention (for the system configuration of the capsule endoscope 100, refer to FIG. 8).

In this capsule endoscope examination instrument 200a, the control unit 5 can be equipped with a control signal processing/transmission unit 5a, a received power indication signal reception/processing unit 5b, an image signal reception/processing unit 5c, a manipulator control unit 5d, and a storage disc 5e. The transmission/reception antenna 6, the manipulation unit 7, the display unit 8, and the manipulators 3a and 3b can be connected to the control unit 5.

In the manipulation unit 7, a control signal that has been input through a keyboard, a mouse, a switch, or a lever can be encoded by the control signal processing/transmission unit 5a and sent from the transmission/reception antenna 6, whereby the camera 14, the illumination devices 15, the liquid chemical supply device 40, the self-propulsion drive device 50, the microhand device 60, etc. of the capsule endoscope 100 can be manipulated remotely. An image signal that is transmitted from the capsule endoscope 100 by a wireless communication can be received by the transmission/reception antenna 6, and decoded by the image signal reception/processing unit 5c into image data, which can be displayed on the display unit 8 or stored in the storage disc 5e.

A received power indication signal for notification of the magnitude of power received by the capsule endoscope 100 can be received by the transmission/reception antenna 6, decoded by the received power indication signal reception/processing unit 5b, and sent to the manipulator control unit 5d. The manipulator control unit 5d can perform a scan by moving the power-transmission antenna 2a and/or 2b relative to the examination stage 1 and stop the scan at a position where the power-transmission antenna 2a and/or 2b allows the received power indication signal to be larger than or equal to a prescribed value, preferably, take a maximum value. The sentence “the received power indication signal is larger than or equal to the prescribed value” means that it is larger than or equal to necessary received power.

In the above mode, the power-transmission antenna 2a and 2b can be positioned so that a received power indication signal becomes larger than or equal to the prescribed value, preferably, takes a maximum value, irrespective of where the capsule endoscope 100 is located.

However, in this system configuration, it takes a certain time to perform a scan to find a power supply position where to allow a received power indication signal to take a maximum value. Whereas the position of the capsule endoscope 100 is not changed quickly by a vermicular movement of the digestive tract, its posture may vary frequently. Since a scan needs to be performed almost from the start if the posture has been changed by close to 90°, this example configuration tends to be lower in efficiency than the following example configuration 2.

### (Example System Configuration 2)

FIG. 13 is a functional block diagram showing the system configuration of another capsule endoscope examination instrument according to the invention (for the system configuration of the capsule endoscope 100, refer to FIG. 8).

However, in this case, the capsule endoscope 100 is equipped with X-ray markers at at least two position in the longitudinal direction in addition to the system configuration shown in FIG. 8.

This capsule endoscope examination instrument 200b is additionally equipped with an X-ray marker position detector 9 and a power-transmission antenna optimum arrangement calculation unit 5f.

A position and a posture of the capsule endoscope 100 detected by the X-ray marker position detector 9 are input to the antenna optimum arrangement calculation unit 5f, which calculates positions of the power-transmission antennas 2a and 2b where the power-transmission antennas 2a and 2b allow received power to become larger than or equal to a prescribed value, preferably, take a maximum value. The manipulator control unit 5d can set the power-transmission antennas 2a and 2b at optimum positions by moving the manipulators 3a and 3b on the basis of calculation results. The sentence “the received power is larger than or equal to the prescribed value” means that it is larger than or equal to necessary received power.

Although the received power indication signal reception/processing unit 5b which receives and decodes a signal for notification of the magnitude of received power is left as an auxiliary function to enable monitoring of a power reception state, it is not connected to the power-transmission antenna optimum arrangement calculation unit 5f and does not relate to the position control directly (in this mode, the received power indication signal reception/processing unit 5b is not indispensable).

In the above mode, a position and a posture of the capsule endoscope 100 are detected using the X-ray markers and the power-transmission antennas 2a and 2b can be placed so that the received power becomes larger than the prescribed value, preferably a maximum value irrespective of whether the capsule endoscope 100 is located at any position. As a result, even when the capsule endoscope 100 has changed its posture suddenly, stable endoscope examination can be performed by securing necessary received power by moving the power-transmission antennas 2a and 2b efficiently.

### [Power-Transmission Antenna]

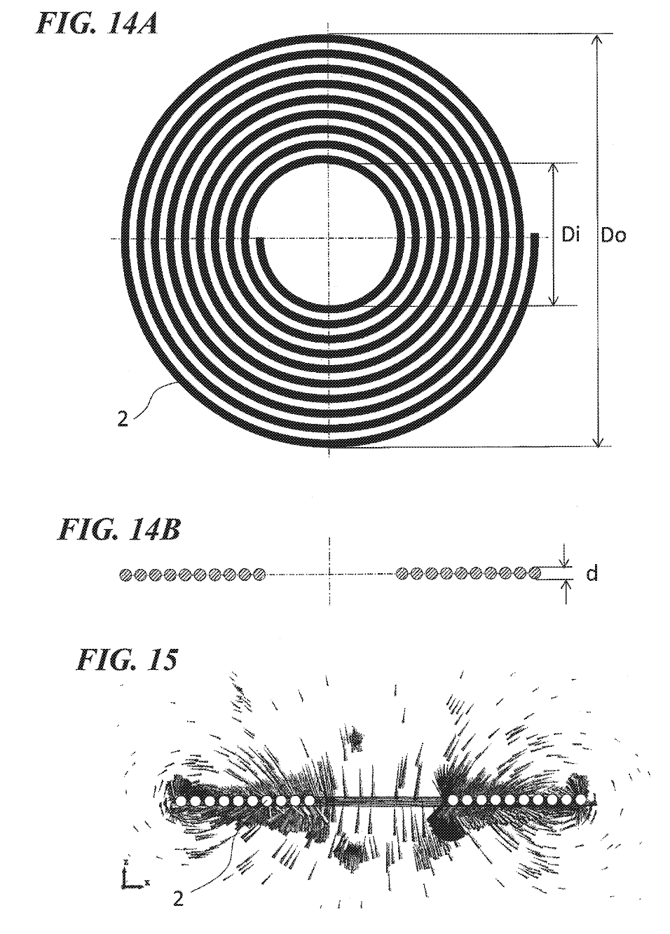

FIGS. 14A and 14B are a schematic plan view and sectional view, respectively, of a power-transmission antenna 2 used in the invention.

The power-transmission antenna 2 is formed by winding a conductor spirally in a plane and has a ring shape having a central hole. When the power-transmission antenna 2 is supplied with AC power, a divergent magnetic field can be formed which diverges outward from the central hole. This mode enables efficient supply of power because the power-transmission antenna 2 can be placed so that magnetic field lines become parallel with the axis of the cylindrical power-reception coil irrespective of where the capsule endoscope 100 is located at any position. In the invention, it is appropriate the size of the power-transmission antenna 2 be approximately the same as the width of a human body; it is preferable that the outer diameter Do be 200 to 500 mm, the inner diameter Di be 40 to 300 mm, and the ratio of the inner diameter Di to the outer diameter Do be 0.2 to 0.6. There are no particular limitations on the wire to constitute the power-transmission antenna 2; common wires can be used in which a copper core wire is covered with an insulating coating layer. However, if the interval between adjoining core wires is too small, it is difficult to form a strong magnetic field due to mutual interference; it is preferable that the gap between an nth turn and an (n+1)th turn be in a range of 0.2d to 2d where d is the diameter of the wire. For example, this condition is satisfied when a wire of 2 mm in outer diameter having a copper core wire of 1 mm in diameter and a thick insulating coating layer is used. It suffices that a wire be wound in one direction, that is, a wire may be wound either clockwise or counterclockwise.

The power-transmission antenna 2 may be such that plural planar, spiral windings of a wire are stacked in such a manner that in a sectional view the wires belonging to the respective layers are arranged in a staggered manner. A strong magnetic field can be generated by increasing the number of turns of the antenna 2; the strength of the magnetic field is proportional to the number of turns.

### [Magnetic Field Generated by Power-Transmission Antenna]

Next, a mode of a magnetic field generated by the above-described antenna (the planar, spiral antenna having a central hole) will be described in a specific manner on the basis of an electromagnetic field simulation result.

An example will be described below in which directions and strengths of a magnetic field to be generated when an energization current of 1 A is caused to flow through a planar, spiral power-transmission antenna having a central hole (Di/Do=(about 80 mm)/(about 240 mm); (number of turns)=10) were calculated.

FIG. 15 is a sectional view of the power-transmission antenna 2 showing directions and strengths of a magnetic field in vectors (cones) on the basis of the simulation result. The direction and the size of each cone represent the direction and the strength of a magnetic field, respectively.

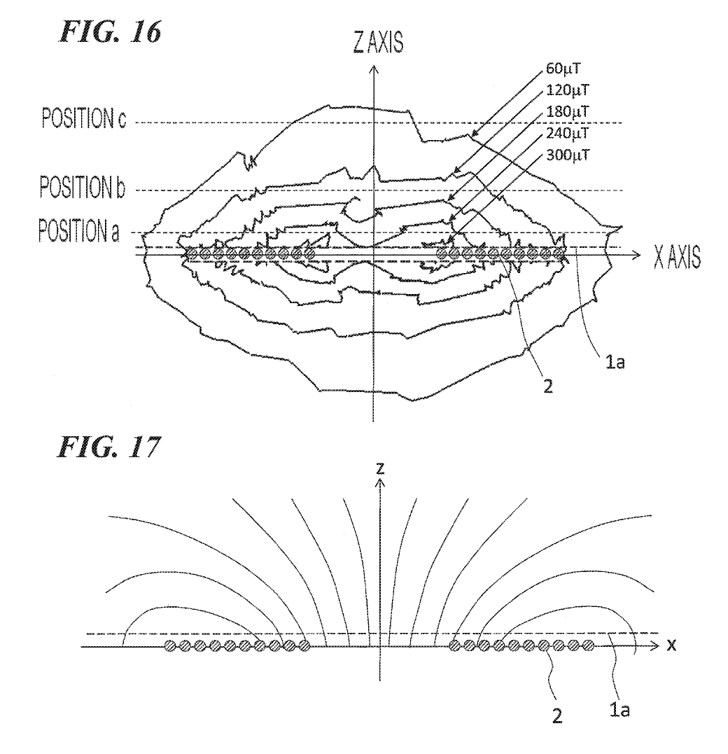

FIG. 16 is a is a sectional view of the power-transmission antenna 2 in which magnetic field strengths are shown as contour lines on the basis of the simulation result. As seen from FIG. 16, the magnetic field is strongest in the vicinity of the innermost part of the conductor of the power-transmission antenna 2 and becomes weaker the position goes away from there, that is becomes weaker gradually as the distance from there increases.

In the X-axis direction in FIG. 16, at position a that is close to the power-transmission antenna 2, the magnetic field is strongest in the vicinity of the inner edge of the power-transmission antenna 2 and becomes weaker gradually as the position comes closer to the position corresponding to the center or the outer edge. At position b that is distant from the power-transmission antenna 2 in the vertical direction approximately by the radius of the inner circumference of the power-transmission antenna 2, the magnetic field is constant from the position corresponding to the center of the power-transmission antenna 2 to a position approximately corresponding to the middle of the ring-shaped portion bounded by the inner edge and the outer edge and becomes weaker gradually as the position comes closer to the position corresponding to the coil outer edge. At position c that is further distant from the power-transmission antenna 2 in the vertical direction, the magnetic field is strongest at the position corresponding to the center of the power-transmission antenna 2 and becomes weaker gradually as the position comes closer to the position corresponding to the coil outer edge. Position b that is a little distant from the power-transmission antenna 2 is not preferable for the supply of power because the magnetic field strength attenuates to about ½ there, in spite of an advantage that the magnetic field uniformity is high there.

It is therefore preferable to set the power-transmission antenna 2 as close to the body of a subject as possible (i.e., priority is given to securing of a strong magnetic field) and to accommodate attenuation of magnetic field strength in the horizontal direction by moving the power-transmission antenna 2 in the horizontal direction.

As shown in FIG. 11, the arrangement that the power-transmission antenna 2a is disposed under the subject placement part 1a and the power-transmission antenna 2b is disposed over a subject is preferable because it enables the following manner of use. That is, when the capsule endoscope 100 is located close to the back of a subject, the power-transmission antenna 2a is set close to the back of a subject. When the capsule endoscope 100 is located close to the abdomen-side surface of a subject, the power-transmission antenna 2b is set so as to be in weak contact with the abdomen of the subject.

FIG. 17 is a sectional view of the power-transmission antenna 2 in which magnetic field lines are drawn on the basis of the simulation result. It is seen that the magnetic field forms loops that flow upward from the central empty region of the power-transmission antenna 2, diverge outward, then change the directions gradually, and finally converge so as to return to the empty region. In the wireless supply of power to the capsule endoscope 100 according to the invention, it is preferable to set the power-transmission antenna 2 so that the axis of the cylindrical power-reception coil 20 coincides with the directions of a magnetic field generated by the power-transmission antenna 2 and the power reception efficiency is thereby maximized.

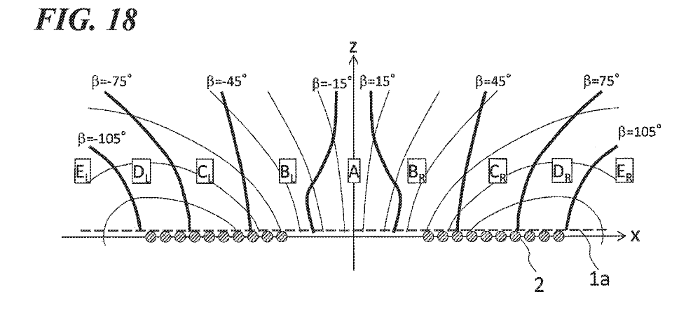

In FIG. 18, isoclinic lines each of which is obtained by connecting spatial points where the inclination angle β of a magnetic field line with respect to the vertical line (Z axis) is the same are drawn in thick lines. Using the isoclinic lines, the space can be classified into regions E_{L} (β<−105°), D_{L} (105°≦−β<75°), C_{L} (−75°≦β<−45°), B_{L} (−45°≦β<−15°), A (−15°≦β<15°), B_{R} (15°≦β<45°), C_{R} (45°≦β<75°), D_{R} (75°≦β<105°), and E_{R} (β≧105°). The thus-defined spatial regions and their symbols will be used for facilitating understanding in later describing a method for placing the power-transmission antenna at a most appropriate position.

### [Power-Transmission Antenna Placing Method]

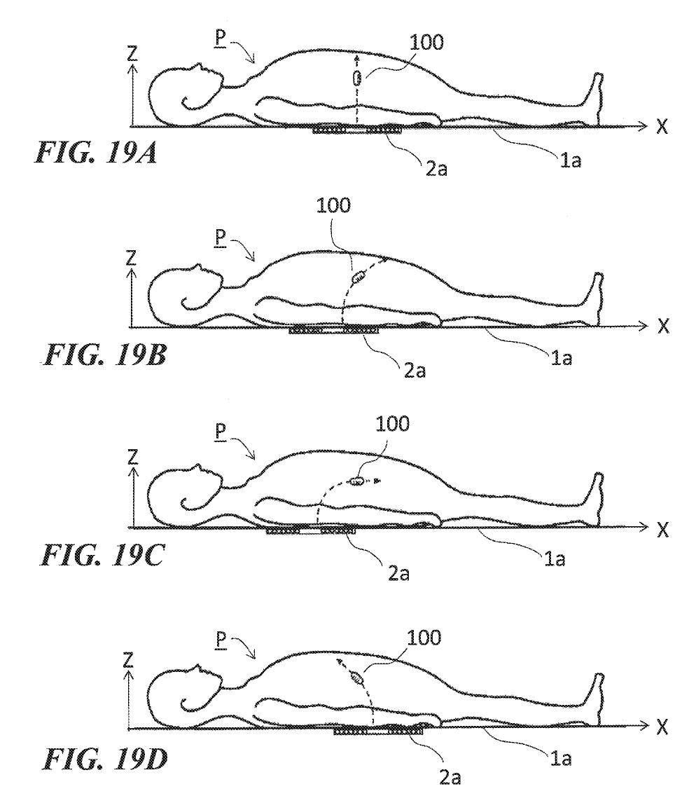

First, a power-transmission antenna placing method will be described briefly using a two-dimensional model. FIGS. 19A to 19D show a subject P who lies on his or her back on a subject placement surface 1a of an examination stage, the capsule endoscope 100, and the power-transmission antenna 2a which is disposed under the subject placement surface 1a. Whereas the relative positioning between the capsule endoscope 100 and the subject P is kept the same, the posture of the capsule endoscope 100 varies from one drawing to another. Now, for the sake of convenience, the Z axis that is perpendicular to the subject placement surface 1a and the X axis that extends from the head to the foot are set and no consideration will be given to the Y axis that is perpendicular to the paper surface. Only related one of magnetic field lines formed by the power-transmission antenna 2a is drawn by a broken line.

In FIG. 19A, the axis of the cylindrical capsule endoscope 100 (i.e., the axis of the cylindrical power-reception coil 20) is parallel with the Z axis. In this case, magnetic field lines extending in the vertical direction can be made parallel with the axis of the cylindrical capsule endoscope 100 by setting the power-transmission antenna 2a approximately directly under the capsule endoscope 100. In FIG. 19B, the axis of the cylindrical capsule endoscope 100 is inclined rightward with respect to the Z axis. In this case, magnetic field lines that are inclined rightward can be made parallel with the axis of the cylindrical capsule endoscope 100 by setting the power-transmission antenna 2a at a position that is closer to the head of the subject P. In FIG. 19C, the axis of the cylindrical capsule endoscope 100 is perpendicular to the Z axis. In this case, horizontal magnetic field lines can be made parallel with the axis of the cylindrical capsule endoscope 100 by setting the power-transmission antenna 2a at a position that is even closer to the head of the subject P. In FIG. 19D, the axis of the cylindrical capsule endoscope 100 is inclined leftward with respect to the Z axis. In this case, magnetic field lines that are inclined leftward can be made parallel with the axis of the cylindrical capsule endoscope 100 by setting the power-transmission antenna 2a at a position that is closer to the foot of the subject P.

Next, a description will be made of a method for placing the power-transmission antenna at a most appropriate position three-dimensionally according to a position and a posture of the capsule endoscope 100.

For three-dimensional consideration, the Y axis that extends in the left-right direction of the body of the subject P is set in addition to the X axis that extends from the head top to the foot and the Z axis that is perpendicular to the subject placement surface 1a of the examination stage. In this coordinate system, the posture of the capsule endoscope 100 is expressed by the azimuth angle αw with respect to the X axis of a projected image obtained by projecting the axis of the cylindrical capsule endoscope 100 onto the XY plane and the inclination angle βc of the axis of the cylindrical capsule endoscope 100 with respect to the Z axis.

A position (Xc, Yc, Zc) of the center of the power-reception coil 20 and a posture (αc, βc) of the power-reception coil 20 can be known using the capsule endoscope examination instrument 200b which is equipped with the X-ray marker position detector 9.

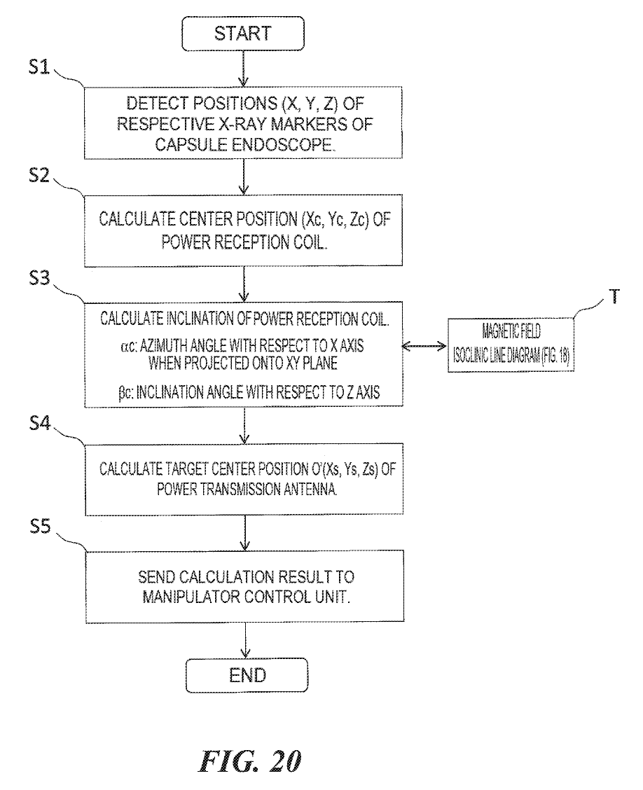

FIG. 20 is a flowchart illustrating a method for setting the power-transmission antenna 2 at a most appropriate position.

At step S1, positions of the X-ray markers of the capsule endoscope 100 is detected by the X-ray marker position detector 9. Pieces of position information of the respective X-ray markers can be input to the antenna optimum arrangement calculation unit 5f of the control unit 5 of the capsule endoscope examination instrument 200b from the X-ray marker position detector 9.

At step S2, the antenna optimum arrangement calculation unit 5f calculates a center position (Xc, Yc, Zc) of the power-reception coil 20 on the basis of the positions of the X-ray markers.

At step S3, the antenna optimum arrangement calculation unit 5f calculates an inclination angle of the power-reception coil 20 on the basis of the positions of the X-ray markers. Since the axis of the cylindrical capsule endoscope 100 coincides with the axis of the cylindrical power-reception coil 20, an inclination of the power-reception coil 20 can be calculated easily below on the basis of an inclination of the capsule endoscope 100. The two angular parameters αc and βc exist as parameters that represent the inclination of the power-reception coil 20. The parameter αc is the azimuth angle of the power-reception coil 20 with respect to the X axis when it is projected onto the XY plane, and βc is the inclination angle of the power-reception coil 20 with respect to the Z axis.

At step S4, the antenna optimum arrangement calculation unit 5f calculates a target center position O′ of the power-transmission antenna 2.

A procedure for calculating a target center position O′ is as follows.

First, consideration is given to a first movement for registering the center of the power-transmission antenna 2 with the center (Xc, Yc, Zc) of the power-reception coil 20. In a state that the center registration has been made, the three-dimensional space is cut by a plane (having the azimuth angle αc) that is parallel with the axis of the cylindrical power-reception coil 20. A magnetic field isoclinic line diagram as shown in FIG. 18 can be applied to the cutting surface. Then consideration is given to a second movement for moving the power-transmission antenna 2 in the direction (having the azimuth angle αc) of the axis of the cylindrical power-reception coil 20 to a position where the inclination β of the magnetic field coincides with the inclination βc of the axis of the cylindrical power-reception coil 20. The movement of the power-transmission antenna 2 to a target center position O′ (Xs, Ys, Zs) of the power-transmission antenna 2 can be calculated as a combination of the first movement of the second movement.

Where a Zs coordinate of a target center position O′ (Xs, Ys, Zs) is fixed, an Xs coordinate and a Ys coordinate of the target center position O′ are determined uniquely. However, where a Zs coordinate of a target center position O′ (Xs, Ys, Zs) is variable, a target center position O′ is not determined uniquely for βc and plural positions satisfy the conditions. However, changing the Zc coordinate in such direction that the power-transmission antenna 2 goes away from the subject P is not preferable because it weakens the magnetic field. Unless there is a special reason to the contrary, it is better to fix the power-transmission antenna 2 at a position that is as close to the subject P as possible. The special reason means that the body of the subject P is so thick that the capsule endoscope 100 is distant too much from the subject placement surface 1a in the Z direction. However, in this case, it is preferable to use the abdomen-side power-transmission antenna 2b rather than the back-side power-transmission antenna 2a. Thus, practically, there is almost no need for a position adjustment involving the Zc coordinate.

As soon as a target center position O′ of the power-transmission antenna 2 is calculated, at step S5 a calculation result is set to the manipulator control unit 5d and the power-transmission antenna 2a or 2b is moved to the target center position O′ by the manipulator 3a or 3b.

Specific descriptions will be made below by assuming various relative positional relationships in the three-dimensional space.

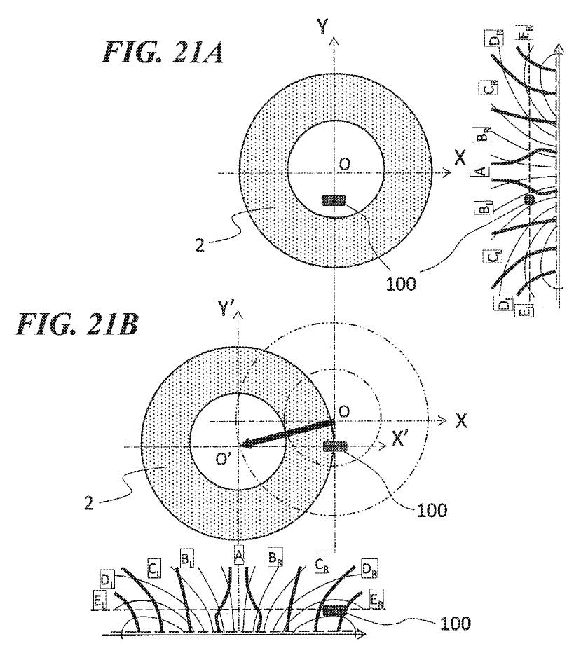

FIG. 21A shows an initial position and posture of the power-reception coil 20 in one mode. The power-reception coil 20 is located on the Y axis and in region B_{L} and is oriented parallel with the X axis (αc=0°) and perpendicular to the Z axis (βc=90°). In the state shown in FIG. 21A, the axis of the cylindrical power-reception coil 20 is perpendicular to magnetic field lines and hence a magnetic flux is hard to enter the inside of the power-reception coil 20 (i.e., the received power is almost equal to zero). In this case, on the computer, first the power-transmission antenna 2 is translated imaginarily in the negative Y-axis direction so that the center (Xs, Ys, Zs) of the power-transmission antenna 2 is registered with the center (Xc, Yc, Zc) of the power-reception coil 20. Since the axis of the power-reception coil 20 is parallel with the X axis, the power-transmission antenna 2 is moved along the X axis until β becomes equal to βc, in other words, the position of the power-reception coil 20 goes into region D_{R}. These two movements are combined together on the computer and, in actuality, the power-transmission antenna 2 is moved along a shortest-distance route. FIG. 21B shows an optimum position of the antenna 2 the movement of which has been completed.

As described above, when the axis of the cylindrical power-reception coil 20 is parallel with a plane that is perpendicular to the axis of the power-transmission antenna 2, the power-transmission antenna 2 is moved so that the capsule endoscope 100 comes to be located near the outer edge of the power-transmission antenna 2 and the axis of the cylindrical power-reception coil 20 is directed in a radial direction of the power-transmission antenna 2.

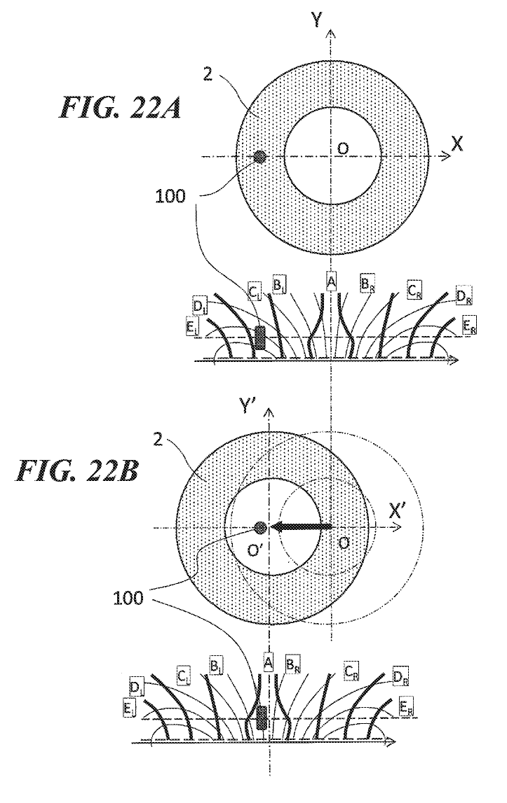

FIG. 22A shows an initial position and posture of the power-reception coil 20 in another mode. The power-reception coil 20 is located on the X axis and in region C_{L} and is oriented parallel with the Z axis (βc=0°). The power reception efficiency is low because the axis of the cylindrical power-reception coil 20 and magnetic field lines form an angle in a range of −75° to −45°. Also in this case, on the computer, the power-transmission antenna 2 is translated imaginarily in the negative X-axis direction so that the center (Xs, Ys, Zs) of the power-transmission antenna 2 is registered with the center (Xc, Yc, Zc) of the power-reception coil 20. In this example, since the power-reception coil 20 enters region A where β is equal to βc, it is no longer necessary to move the power-transmission antenna 2. FIG. 22B shows an optimum position of the antenna 2 the movement of which has been completed.

As described above, when the axis of the cylindrical power-reception coil 20 is parallel with the axis of the power-transmission antenna 2, the power-transmission antenna 2 is moved so that the capsule endoscope 100 comes to be located inside the inner edge of the power-transmission antenna 2.

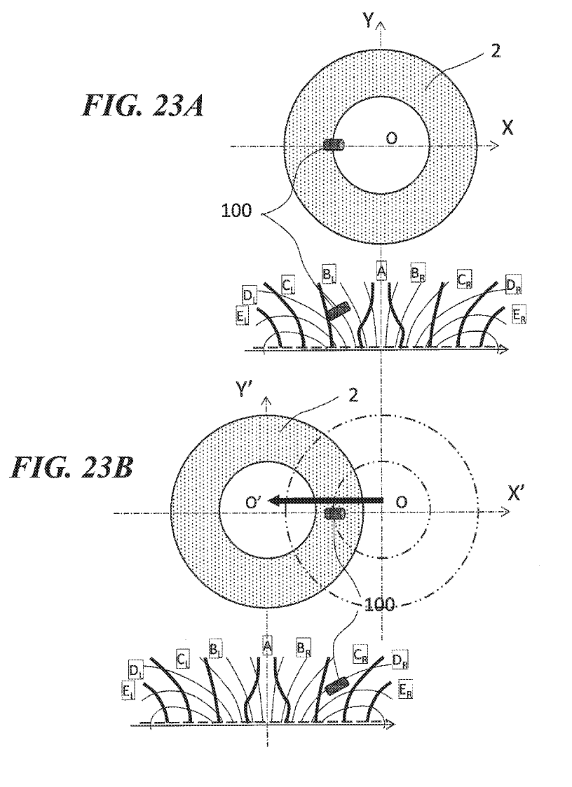

FIG. 23A shows an initial position and posture of the power-reception coil 20 in a still another mode. The power-reception coil 20 is located on the X axis and in region B_{L}. The power-reception coil 20 is oriented parallel with the X axis (αc=0°) but forms an angle 45° with the Z axis (βc=45°). In the state shown in FIG. 23A, the axis of the cylindrical power-reception coil 20 is perpendicular to magnetic field lines and hence a magnetic flux is hard to enter the inside of the power-reception coil 20 (i.e., the received power is almost equal to zero). Also in this case, on the computer, first the power-transmission antenna 2 is translated imaginarily in the negative X-axis direction so that the center (Xs, Ys, Zs) of the power-transmission antenna 2 is registered with the center (Xc, Yc, Zc) of the power-reception coil 20. Since the axis of the power-reception coil 20 is parallel with the X axis, the power-transmission antenna 2 is moved along the X axis until β becomes equal to βc, in other words, the position of the power-reception coil goes into region C_{R}. These two movements are combined together on the computer and, in actuality, the power-transmission antenna 2 is moved along a shortest-distance route. FIG. 23B shows an optimum position of the antenna 2 the movement of which has been completed.

As described above, when the axis of the cylindrical power-reception coil 20 is inclined from a plane that is perpendicular to the axis of the power-transmission antenna 2, the power-transmission antenna 2 is moved so that the capsule endoscope 100 comes to be located in the ring portion between the inner edge and the outer edge of the power-transmission antenna 2 and the axis of the cylindrical power-reception coil 20 is directed in a radial direction of the power-transmission antenna 2.

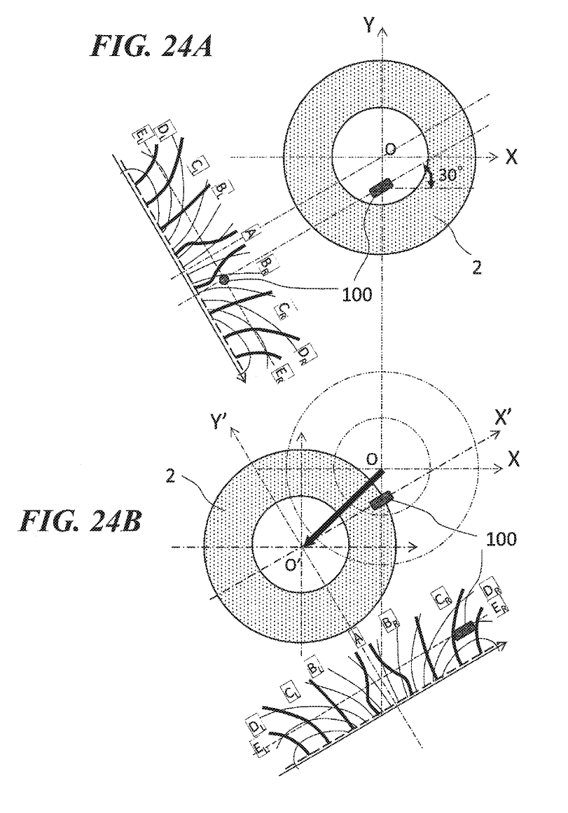

FIG. 24A shows an initial position and posture of the power-reception coil 20 in a still another mode. The power-reception coil 20 is located on the Y axis and in region B_{R}. The power-reception coil 20 forms an angle 30° with the X axis (αc=30°) and is oriented perpendicularly to the Z axis (βc=90°). In the state shown in FIG. 24A, the axis of the cylindrical power-reception coil 20 crosses magnetic field lines at a large angle and hence the received power is almost equal to zero. Also in this case, on the computer, first the power-transmission antenna 2 is translated imaginarily in the negative Y-axis direction so that the center (Xs, Ys, Zs) of the power-transmission antenna 2 is registered with the center (Xc, Yc, Zc) of the power-reception coil 20. Since the axis of the power-reception coil 20 is in the direction that forms the angle 30° with the X axis, the power-transmission antenna 2 is moved in this direction until β becomes equal to βc, in other words, the position of the power-reception coil 20 goes into region D_{R}. These two movements are combined together on the computer and, in actuality, the power-transmission antenna 2 is moved along a shortest-distance route. FIG. 24B shows an optimum position of the antenna 2 the movement of which has been completed.

As described above, when the axis of the cylindrical power-reception coil 20 is parallel with a plane that is perpendicular to the axis of the power-transmission antenna 2, the power-transmission antenna 2 is moved so that the capsule endoscope 100 comes to be located near the outer edge of the power-transmission antenna 2 and the axis of the cylindrical power-reception coil 20 is directed in a radial direction of the power-transmission antenna 2.

Next, a description will be made of a wireless power supply method that increases the power supply amount using the two power-transmission antennas 2a and 2b.

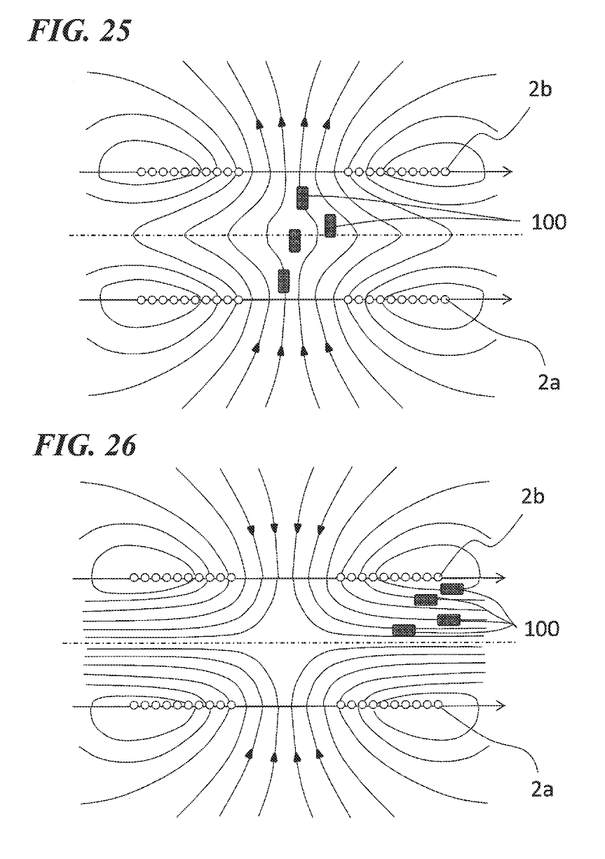

FIG. 25 illustrates a power supply method that is suitable for the capsule endoscope 100 that is oriented perpendicularly to the power-transmission antenna planes. The power-transmission antennas 2a and 2b are formed by winding a wire in the same direction and arranged in such a manner that their axes coincide with each other. By applying AC powers of the same phase to the power-transmission antennas 2a and 2b, a strong, uniform magnetic field can be formed in a wide range around the antenna centers of the space located between the two power-transmission antennas 2a and 2b perpendicularly to the power-transmission antenna planes. Four capsule endoscopes 100 are shown in FIG. 25. All of the four capsule endoscopes 100 are given similarly large power supply efficiency values, which means that stable supply of power is possible in which the power reception efficiency does not vary even if the position of the capsule endoscope 100 is changed a little.

Where the power-transmission antennas 2a and 2b are formed by winding a wire in opposite directions, the same results as described above are obtained by forming magnetic fields in the same direction by deviating the phases of AC powers applied to them by a half cycle.

As described above, when the axis of the cylindrical power-reception coil 20 is parallel with the axis of each of the power-transmission antennas 2a and 2b, it is appropriate to arrange the first power-transmission antenna and the second power-transmission antenna coaxially, move them so that the capsule endoscope 100 is placed inside the inner edges of the respective power-transmission antennas, and perform wireless supply of power in such a manner that the first power-transmission antenna and the second power-transmission antenna generate magnetic fields in the same direction.

FIG. 26 illustrates a power supply method that is suitable for the capsule endoscope 100 that is oriented parallel with the power-transmission antenna planes. The power-transmission antennas 2a and 2b are formed by winding a wire in the same direction and arranged in such a manner that their axes coincide with each other. By applying AC powers whose phases are deviated from each other by a half cycle to the power-transmission antennas 2a and 2b, strong, uniform magnetic fields that are parallel with the power-transmission antenna planes can be formed in wide regions, corresponding to the antenna ring portions, of the space located between the two power-transmission antennas 2a and 2b. Four capsule endoscopes 100 are shown in FIG. 26. All of the four capsule endoscopes 100 are given similarly large power supply efficiency values, which means that stable supply of power is possible in which the power reception efficiency does not vary even if the position of the capsule endoscope 100 is changed a little.

Where the power-transmission antennas 2a and 2b are formed by winding a wire in opposite directions, the same results as described above are obtained by forming magnetic fields in opposite directions by setting the phases of AC powers applied to them identical.

As described above, when the axis of the cylindrical power-reception coil 20 is parallel with a plane that is perpendicular to the axis of each of the power-transmission antennas 2a and 2b, it is appropriate to arrange the first power-transmission antenna and the second power-transmission antenna coaxially, move them so that the capsule endoscope 100 is placed in a region between the ring portions each of which is bounded by the inner edge and the outer edge of a power-transmission antenna, and perform wireless supply of power in such a manner that the first power-transmission antenna and the second power-transmission antenna generate magnetic fields in opposite directions.

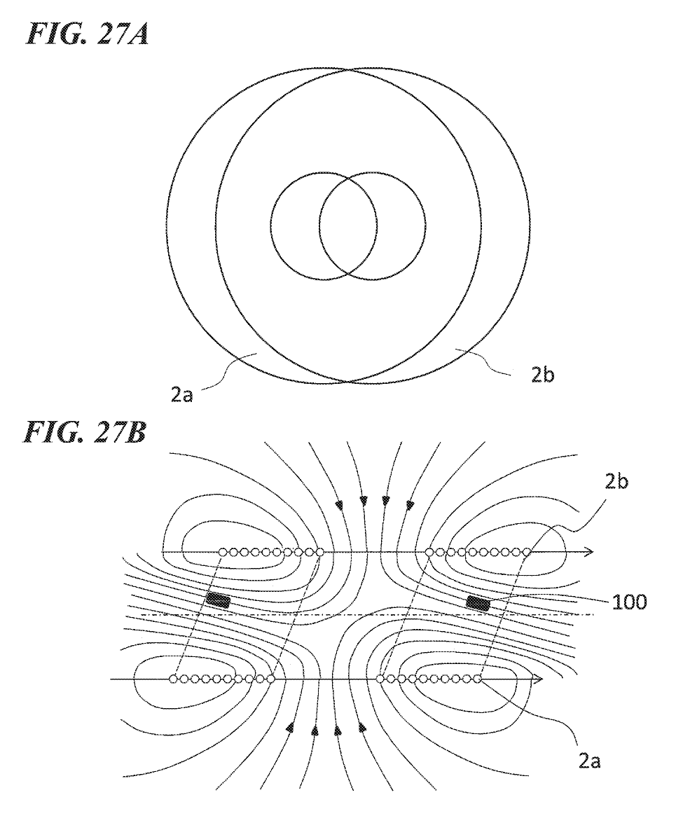

FIGS. 27A and 27B illustrate a power supply method that is suitable for the capsule endoscope 100 that is oriented so as to be inclined with respect to the power-transmission antenna planes. The power-transmission antennas 2a and 2b are formed by winding a wire in the same direction and deviated from each other in such a manner that their central holes overlap with each other. In FIGS. 27A and 27B, the power-transmission antennas 2a and 2b are deviated from each other by ½ of their inner diameter Di. By applying AC powers whose phases are deviated from each other by a half cycle to the power-transmission antennas 2a and 2b, strong, uniform magnetic fields that are inclined with respect to the power-transmission antenna planes can be formed in wide regions, corresponding to the antenna ring portions, of the space located between the two power-transmission antennas 2a and 2b.

Where the power-transmission antennas 2a and 2b are formed by winding a wire in opposite directions, the same results as described above are obtained by forming magnetic fields in opposite directions by setting the phases of AC powers applied to them identical.

As described above, when the axis of the cylindrical power-reception coil 20 is inclined with respect to a plane that is perpendicular to the axes of the power-transmission antennas 2a and 2b, it is appropriate to deviate the first power-transmission antenna and the second power-transmission antenna from each other so that their central holes overlap with each other and the directions of magnetic fields generated by them are thereby inclined in the same direction as the axis of the cylindrical power-reception coil 20, and perform wireless supply of power in such a manner that the first power-transmission antenna and the second power-transmission antenna generate magnetic fields in opposite directions.

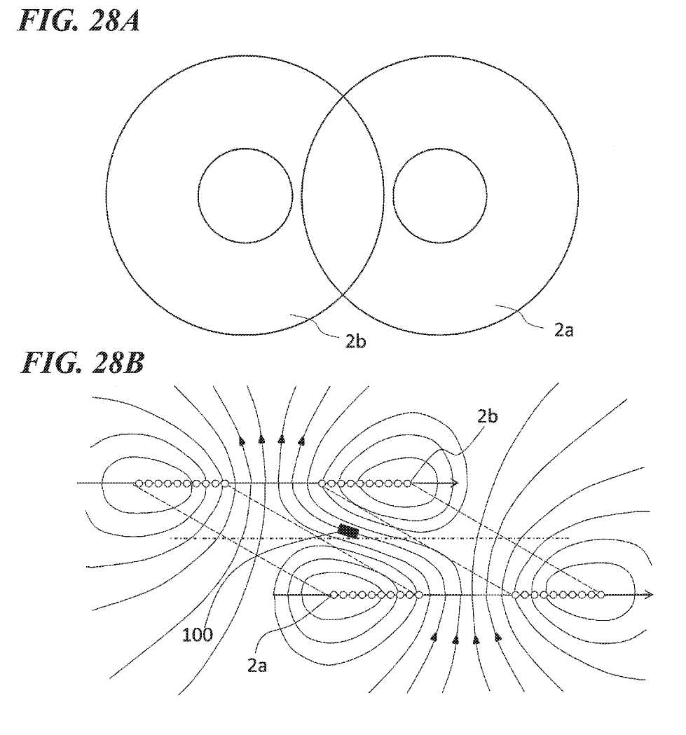

FIGS. 28A and 28B illustrate another power supply method that is suitable for the capsule endoscope 100 that is oriented so as to be inclined with respect to the power-transmission antenna planes. The power-transmission antennas 2a and 2b are formed by winding a wire in the same direction and deviated so that their central holes do not overlap with each other. In FIGS. 28A and 28B, the power-transmission antennas 2a and 2b are deviated from each other by more than ½ of their outer diameter Do. By applying AC powers having the same phase to the power-transmission antennas 2a and 2b, strong, uniform magnetic fields that are inclined with respect to the power-transmission antenna planes can be formed in wide regions, corresponding to the antenna ring portions, of the space located between the two power-transmission antennas 2a and 2b.

Where the power-transmission antennas 2a and 2b are formed by winding a wire in opposite directions, the same results as described above are obtained by forming magnetic fields in the same direction by deviating the phases of AC powers applied to them by a half cycle.

### EXAMPLES

In Examples, the relationship between the power reception efficiency and the makeups of the power-reception coil and the magnetic body will be described in detail on the basis of evaluation data.

### Example 1

In the capsule endoscope 100c shown in FIGS. 4A and 4B (external coil type), the thickness d₀ of the capsule barrel 13c was set at 10 mm, the thickness d₁ of the core wall of the recess 13c1 was set at 0.3 mm, the relative permeability of the magnetic body 30b was set at 130, the thickness d₂ of the magnetic body 30c was set at 0.2 mm, the thickness d₃ of the power-reception coil 20c formed by winding an enamel wire of 0.15 mm in diameter in two layers was set at 0.3 mm, and the coil length I of the power-reception coil 20c was set at 5 mm.

### Example 2

The makeups were the same as in Example 1 except that the coil length I of the power-reception coil 20c was set at 4 mm.

### Example 3

The makeups were the same as in Example 1 except that the coil length I of the power-reception coil 20c was set at 6.5 mm.

### Example 4

The makeups were the same as in Example 1 except that the thickness d₂ of the magnetic body 30c was set at 0.15 mm.

### Example 5

The makeups were the same as in Example 1 except that the thickness d₂ of the magnetic body 30c was set at 0.3 mm.

### Example 6

The makeups were the same as in Example 1 except that the relative permeability of the magnetic body 30c was set at 30.

### Example 7

The makeups were the same as in Example 1 except that the relative permeability of the magnetic body 30c was set at 100.

### Example 8

The makeups were the same as in Example 1 except that the relative permeability of the magnetic body 30c was set at 200.

### Example 9

The makeups were the same as in Example 1 except that the relative permeability of the magnetic body 30c was set at 1,000.

### Example 10

The capsule endoscope 100a shown in FIG. 1 (internal coil type) was employed as a sample. The relative permeability of the magnetic body 30a was set at 100 and the thickness of the magnetic body 30a was set at 0.2 mm. The thickness of the power-reception coil 20a was set at 0.3 mm and the coil length of the power-reception coil 20a was set at 10.0 mm.

### Comparative Example 1

In the capsule endoscope 100c shown in FIG. 2 (external coil type), the setups were the same as in Example 1 except that the magnetic body 30c was not used.

### [Test 1]

In Test 1, an inductance, a resistance, an impedance, and a Q value of each of Examples 1-3 and Example 10 were measured using an LCR meter at a frequency 200 kHz.

As seen from Table 1, it can be said that Example 10 (internal coil type) is approximately equivalent to Example 1 (external coil type; coil length: 4 mm) in coil performance. In Examples of the external coil type, the Q value increased and the coil performance was thus improved as the coil length I became longer.

TABLE 1

| | Coil length I (mm) | | | |
|---|---|---|---|---|
| | | External type | | |
| | Internal type | 4 | 5 | 6.5 |
| Inductance L (mH) | 0.0413 | 0.0426 | 0.0673 | 0.0895 |
| Resistance R (Ω) | 1.659 | 1.737 | 2.285 | 2.545 |
| Impedance Z (Ω) | 51.88 | 53.578 | 84.64 | 112.47 |
| Q value | 31.26 | 30.85 | 37.07 | 44.24 |

### [Test 2]

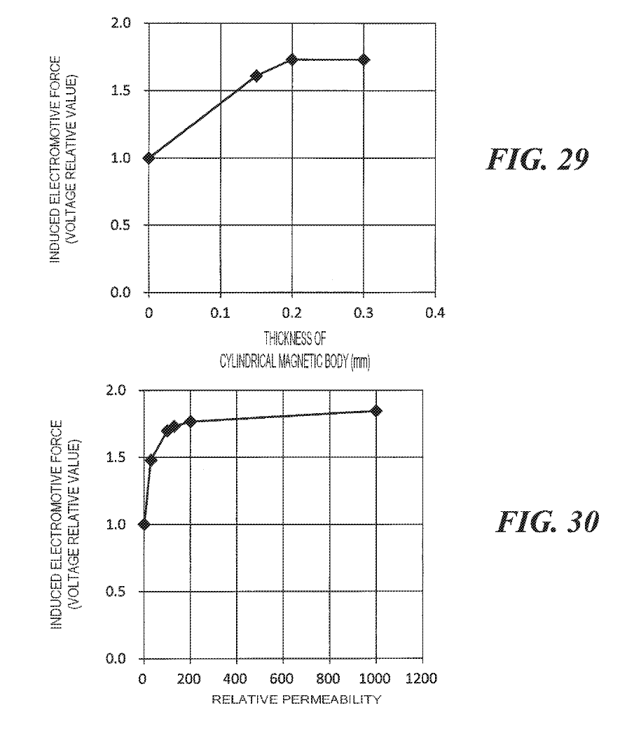

In Test 2, the magnitudes of induced electromotive forces of Examples 1, 4, and 5 (external coil type) and Comparative Example 1 having no magnetic body 30c were compared using ratios of voltages to a result (regarded as “1”) of Comparative Example 1. As seen from FIG. 29, the induced electromotive force increased as the cylindrical magnetic body 30c became thicker but this effect was almost saturated when the thickness was greater than or equal to 0.2 mm.

### [Test 3]

In Test 3, the magnitudes of induced electromotive forces of Examples 1 and 6-9 (external coil type) and Comparative Example 1 having no magnetic body 30c were compared using ratios of voltages to a result (regarded as “1”) of Comparative Example 1. As seen from FIG. 30, the induced electromotive force increased as the permeability of the cylindrical magnetic body 30c was made larger but this effect was almost saturated when the permeability was larger than or equal to 130.

### DESCRIPTION OF SYMBOLS

1: Examination stage
1a: Subject placement surface of examination stage
2, 2a, 2b: Power-transmission antenna
3a, 3b: Manipulator
4: AC power source
5: Control unit
6: Transmission/reception antenna
7: Manipulation unit
8: Display unit
9: X-ray marker position detector
11a, 11b, 11c, 11d: Capsule
12: Tip cover
13a, 13b, 13c, 13d: Capsule barrel
13c1: Ring-shaped recess formed along outer circumference of capsule barrel
13d1: Ring-shaped recess formed along outer circumference of hemispherical portion of capsule barrel
14: Camera
15: Illumination device
16: Semiconductor device
17, 18a, 18b: Electronic circuit board
19: Partition wall
20, 20a, 20b, 20c, 20d: Power-reception coil
21: Coated wire
22c1, 22c2, 22d1, 22d2: Through-hole formed through capsule barrel
23c, 23d: Coating layer covering power-reception coil
24: Cap
25: Resonance capacitor
30a, 30b, 30c, 30d: Magnetic body
40a, 40b: Liquid chemical supply device
41a, 41c: Liquid chemical tank
42: Liquid chemical suction pipe
43: Electromotive valve or pump
44: Liquid chemical emission opening
50: Self-propulsion drive device
60: Microhand device
61: Shape-memorized spring
62: Ceramic heater
63: Scissors
64: Spring
70: X-ray marker
80: Capacitor
90: Vibrator
100, 100a, 100b, 100c, 100d: Capsule endoscope
200, 200a, 200b: Capsule endoscope examination instrument
P: Subject
Q: Tubular organ
R: Sample

1. A generally cylindrical capsule endoscope to be used for diagnosing the condition of the inside of a tubular organ such as a digestive tract by going into the tubular organ, the capsule endoscope including:
a camera which shoots the inside of the tubular organ;
a transceiver which performs a wireless communication with the outside;
a cylindrical power-reception coil which receives power that is supplied from an external power-transmission antenna via a magnetic flux;
a self-propulsion drive device which causes the capsule endoscope to move along the inside of the tubular organ; and
a generally cylindrical capsule which houses the above components,
wherein a magnetic body is disposed adjoining an inner circumferential surface of the power-reception coil,
wherein the self-propulsion drive device has a coil and a magnet, and
wherein the self-propulsion drive device is disposed in series to the power-reception coil in an axial direction of the capsule so as not to be located inside the power-reception coil.

2. The capsule endoscope of claim 1,
wherein a central portion and both end portions of the capsule are cylindrical and hemispherical, respectively, an outer circumferential surface of the cylindrical portion is formed with a ring-shaped recess, the magnetic body is disposed at the bottom of the recess, the power-reception coil is disposed adjoining an outer circumferential surface of the magnetic body, an outer circumferential surface of the power-reception coil is coated with a coating layer, and the magnetic body and the power-reception coil are housed within a wall thickness of the capsule.

3. (canceled)

4. The capsule endoscope of claim 2,
wherein the capsule is 0.5 to 1.0 mm in thickness, the magnetic body is one formed by curling a resin sheet of 100 to 130 in relative permeability and 0.2 to 0.3 mm in thickness containing a ferromagnetic material, and the power-reception coil is a cylindrical coil of 4 to 6.5 mm in coil length formed by winding a coated wire of 0.10 to 0.15 mm in outer diameter around an outer circumferential surface of the magnetic body in two layers.

5. The capsule endoscope of claim 1,
wherein a curled electronic circuit board which controls installed devices is disposed in the capsule.

6. The capsule endoscope of claim 1,
wherein a central portion and both end portions of the capsule are cylindrical and hemispherical, respectively, an outer circumferential surface of a hemispherical portion that is opposite to an end portion where the camera is disposed is formed with a ring-shaped recess, the magnetic body is disposed at the bottom of the recess, the power-reception coil is disposed adjoining an outer circumferential surface of the magnetic body, and the magnetic body and the power-reception coil are housed in the hemispherical portion of the capsule.

7. The capsule endoscope of claim 6,
wherein the magnetic body is one formed by curling a resin sheet of 100 to 130 in relative permeability and 0.1 to 0.5 mm in thickness containing a ferromagnetic material, and the power-reception coil is a cylindrical coil of 3 to 4 mm in coil length formed by winding a coated wire of 0.10 to 0.15 mm in outer diameter around an outer circumferential surface of the magnetic body in three or more layers.

8. The capsule endoscope of claim 1,
wherein a liquid chemical supply device is disposed inside the power-reception coil, and includes:
a non-metal liquid chemical tank;
an electromotive valve or pump which is connected to the liquid chemical tank and driven by power received by the power-reception coil; and
a liquid chemical emission opening which is formed at an end portion of the capsule.

9. The capsule endoscope of claim 1,
wherein a microhand device is disposed inside the power-reception coil, and device includes:
a resin shape-memorized spring which is memorized with an elongated shape at a high temperature and housed in a compressed state at low temperatures;
a ceramic heater which heats the resin shape-memorized spring when driven by power received by the power-reception coil; and
non-metal (resin or ceramic) scissors which are attached to the tip of the shape-memorized spring,
wherein the scissors project from an opening of an end portion of the capsule and open as a result of elongation of the shape-memorized spring that is caused by energization of the ceramic heater, and
wherein when the ceramic heater is deenergized, the shape-memorized spring cools and, during that course, the scissors are pulled back and closed as they come to be confined in the opening of the end portion of the capsule.

10. A capsule endoscope examining method including:
supplying power intermittently to the power-reception coil of the capsule endoscope of claim 1;
detecting a start and an end of a period when the power-reception coil is not supplied with power by a power measurement unit which measures the magnitude of power being received by the power-reception coil or a time measurement unit which operates in synchronism with supply of power to the power-reception coil; and
performing a wireless communication with the outside by the transceiver in the period when the power-reception coil is not supplied with power.

11. A capsule endoscope examination instrument which uses
a capsule endoscope including:
a camera which shoots the inside of a tubular organ;
a transceiver which performs a wireless communication with the outside;
a cylindrical power-reception coil which receives power that is supplied from an external power-transmission antenna via a magnetic flux;
a self-propulsion drive device which causes the capsule endoscope to move along the inside of the tubular organ; and
a generally cylindrical capsule which houses the above components,
wherein a magnetic body is disposed adjoining an inner circumferential surface of the power-reception coil,
wherein the self-propulsion drive device has an electromagnet and a permanent magnet, and
wherein the self-propulsion drive device is disposed in series to the power-reception coil in an axial direction of the capsule so that the permanent magnet is not located inside the power-reception coil,
the capsule endoscope examination instrument including:
the capsule endoscope; and
a power-transmission antenna or antennas which supply power wirelessly to the power-reception coil of the capsule endoscope,
wherein the power-reception coil comprises a cylindrical coil, and
wherein the power-transmission antenna or antennas are ones formed by winding a conductor into a planar spiral.

12. The capsule endoscope examination instrument of claim 11,
wherein the capsule endoscope further includes a transmission unit which measures the magnitude of received power and communicates the magnitude of the received power wirelessly
wherein the power-transmission antenna or antennas are disposed under and/or over a subject placement part of an examination stage on which a subject is placed so as to be movable relative to the examination stage,
wherein the capsule endoscope examination instrument further includes:
a receiving unit which receives a signal from the transmission unit of the capsule endoscope; and
a power-transmission antenna position controller which arranges the power-transmission antenna or antennas onto a position or positions where the received power becomes larger than or equal to a prescribed value, through a scan by moving the power-transmission antenna or antennas relative to the examination stage.

13. The capsule endoscope examination instrument of claim 11,
wherein the capsule endoscope further includes a detector which detects a position and a posture of the capsule,
wherein the power-transmission antenna or antennas are disposed under and/or over a subject placement part of the an examination stage on which a subject is placed so as to be moveable independently relative to the examination stage, and
wherein the capsule endoscope examination instrument further includes:
a receiving unit which receives a signal from a transmission unit of the capsule endoscope;
a position determinator which determines a position or positions of the power-transmission antenna or antennas where the power-transmission antenna or antennas allow the received power to be larger than or equal to a prescribed value on the basis of the position and the posture of the capsule endoscope detected by the detector; and
a power-transmission antenna position controller which moves the power-transmission antenna or antennas on the basis of a result obtained by the position determinator.

14. The capsule endoscope examination instrument of claim 13,
wherein the power-transmission antenna is formed by wind the conductor into the planar spiral to have a circular ring shape having a central hole, and is disposed under or over the subject placement part of the examination stage so that it is movable relative to the examination stage and the axis of the circular ring is perpendicular to the examination stage, and
wherein the position determinator and the position controller control such that,
if the axis of the power-reception coil is parallel with the axis of the circular ring of the power-transmission antenna,
the power-transmission antenna is moved so that the capsule endoscope comes to be located inside an inner edge of the power-transmission antenna,
if the axis of the power-reception coil is parallel with a plane that is perpendicular to the axis of the circular ring of the power-transmission antenna,
the power-transmission antenna is moved so that the capsule endoscope comes to be located near an outer edge of the power-transmission antenna and the axis of the power-reception coil is directed in a radial direction of the power-transmission antenna, and
if the axis of the power-reception coil is inclined with respect to a plane that is perpendicular to the axis of the circular ring of the power-transmission antenna,
the power-transmission antenna is moved so that the capsule endoscope comes to be located in a region of the circular ring between the inner edge and the outer edge of the power-transmission antenna and the axis of the power-reception coil is directed in the radial direction of the power-transmission antenna.

15. The capsule endoscope examination instrument of claim 13,
wherein the power-transmission antennas are formed by winding the conductor into the planar spiral to have a circular ring shape having a central hole, and include a first power-transmission antenna and a second power-transmission antenna that are disposed under and over the subject placement part of the examination stage, respectively, so that they are movable relative to the examination stage and the axis of the circular ring is perpendicular to the examination stage, and
wherein the position determinator and the position controller control such that,
if the axis of the power-reception coil is parallel with the axis of the circular ring of each of the first and second power-transmission antennas,
the first and second power-transmission antennas are arranged coaxially,
moved so that the capsule endoscope comes to be located inside an inner edge of each of the first and second power-transmission antennas, and supplied with power wirelessly so as to generate respective magnetic fields that are in the same direction,
if the axis of the power-reception coil is parallel with a plane that is perpendicular to the axis of the circular ring of each of the first and second power-transmission antennas,
the first and second power-transmission antennas are arranged coaxially,
moved so that the capsule endoscope comes to be located in a region between the respective circular rings bounded by the inner edges and the outer edges of the first and second power-transmission antennas, and supplied with power wirelessly so as to generate respective magnetic fields that are in opposite directions, and
if the axis of the power-reception coil is inclined with respect to a plane that is perpendicular to the axis of the circular ring of each of the first and second power-transmission antennas,
the first and second power-transmission antennas are deviated from each other so that their central holes overlap with each other, moved so that the capsule endoscope comes to be located in a region of an overlap of the circular rings of the first and second power-transmission antennas, and supplied with power wirelessly so as to generate respective magnetic fields that are in opposite directions, or
the first and second power-transmission antennas are deviated from each other so that their central holes do not overlap with each other, moved so that the capsule endoscope comes to be located in a region of an overlap of the circular rings of the first and second power-transmission antennas, and supplied with power wirelessly so as to generate respective magnetic fields that are in the same direction.

16. The capsule endoscope of claim 6,
wherein the power-reception coil is formed by winding a coated wire on an outer circumferential surface of the magnetic body, such that the number of winding turns is large at an end of the capsule where the camera is disposed, and becomes lesser toward an opposite end to the end where the camera is disposed.
